SEQUENCE LISTING (20 sequences)

SEQ ID NO: 1 - RNA, 31 nt, Artificial Sequence
  Synthetic construct
  misc_feature (1)..(31): all nucleotides 2′-MeO protected
gcagcacugugauguugacucuuggcgcugc

SEQ ID NO: 2 - RNA, 33 nt, Artificial Sequence
  Synthetic construct
  misc_feature (1)..(33): all nucleotides2′-MeO protected
ccgguauuguccuucgucucuguuuuugaccgg

SEQ ID NO: 3 - DNA, 30 bp, Artificial Sequence
  Synthetic construct
gcagctctgtctttctttggtctgcgctgc

SEQ ID NO: 4 - DNA, 24 bp, Artificial Sequence
  Synthetic construct
ttgcagaccaaagaaagacagatt

SEQ ID NO: 5 - RNA, 34 nt, Artificial Sequence
  Synthetic construct
  misc_feature (1)..(34): all nucleotides2′-MeO protected
gcagcugcaacucaguuccaucauuacgggcugc

SEQ ID NO: 6 - RNA, 34 nt, Artificial Sequence
  Synthetic construct
  misc_feature (1)..(34): all nucleotides2′-MeO protected
gcucguuugaccuugcuuccuggaaagaucgagc

SEQ ID NO: 7 - DNA, 24 bp, Artificial Sequence
  Synthetic construct
gccaagagtcaacatcacagtgct

SEQ ID NO: 8 - DNA, 24 bp, Artificial Sequence
  Synthetic contruct
ccgtaatgatggaactgagttgca

SEQ ID NO: 9 - DNA, 24 bp, Artificial Sequence
  Synthetic construct
atctttccaggaagcaaggtcaaa

SEQ ID NO: 10 - RNA, 33 nt, Artificial Sequence
  Synthetic construct
  misc_feature (1)..(33): all nucleotides2′-MeO protected
ccgguuuaguuccuguucuguugucuucaccgg

SEQ ID NO: 11 - RNA, 30 nt, Artificial Sequence
  Synthetic construct
  misc_feature (1)..(30): all nucleotides2′-MeO protected
gcagcucugucuuucuuuggucugcgcugc

SEQ ID NO: 12 - DNA, 27 bp, Artificial Sequence
  Synthetic construct
ttgccaagagtcaacatcacagtgctt

SEQ ID NO: 13 - DNA, 27 bp, Artificial Sequence
  Synthetic construct
ttgccaagagtcaacatcacagtgctt

SEQ ID NO: 14 - DNA, 28 bp, Artificial Sequence
  Synthetic construct
ttatctttccaggaagcaaggtcaaatt

SEQ ID NO: 15 - DNA, 27 bp, Artificial Sequence
  Synthetic construct
ttgaagacaacagaacaggaactaatt

SEQ ID NO: 16 - DNA, 27 bp, Artificial Sequence
  Synthetic construct
ttgaagacaacagaagaggaactaatt

SEQ ID NO: 17 - RNA, 34 nt, Artificial Sequence; identical to SEQ ID NO: 5
  Synthetic construct
  misc_feature (1)..(34): all nucleotides2′-MeO protected

SEQ ID NO: 18 - DNA, 23 bp, Artificial Sequence
  Synethtic construct
caaaaacagagacgaaggacaat

SEQ ID NO: 19 - DNA, 20 bp, Artificial Sequence
  Synthetic construct
gcagaccaaagaaagacaga

SEQ ID NO: 20 - DNA, 20 bp, Artificial Sequence
  Synthetic construct
gcagaccgaagaaagacaga

(19) United States
(12) Patent Application Publication
(10) Pub. No.: US 2022/0195500 A1
(43) Pub. Date: Jun. 23, 2022

(54) PROBES FOR DETECTING RNA AND METHODS OF USE THEREOF

(71) Applicant: VANDERBILT UNIVERSITY, Nashville, TN (US)

(72) Inventors: Imam UDDIN, Nashville, TN (US); John S. PENN, Nashville, TN (US); Craig L. DUVALL, Nashville, TN (US)

(21) Appl. No.: 17/606,486

(22) PCT Filed: Apr. 24, 2020
(86) PCT No.: PCT/US2020/029698

§ 371 Date: Oct. 26, 2021

Related U.S. Application Data
(60) Provisional application No. 62/839,077, filed on Apr. 26, 2019.

(51) Int. Cl. C12Q 1/6818; A61K 49/00; A61P 43/00; C07H 21/02

(52) U.S. Cl. CPC C12Q 1/6818; A61K 49/0032; A61K 49/0052; A61K 2039/55; A61K 49/0082; A61P 43/00; C07H 21/02; A61K 49/0054

(57) ABSTRACT

The present disclosure relates to probes and methods for detecting nucleic acids, and for detecting and treating neovas-cularization.

### CROSS REFERENCE TO RELATED APPLICATIONS

This application claims the benefit of U.S. Provisional Patent Application Ser. No. 62/839,077, filed Apr. 26, 2019, the disclosure of which is expressly incorporated herein by reference.

### STATEMENT REGARDING FEDERALLY SPONSORED RESEARCH

This invention was made with government support under Grant No. R01 EY023397 and R01 EY029693 awarded by the National Institutes of Health. The government has certain rights in the invention.

### FIELD

The present disclosure relates to probes and methods for detecting nucleic acids, and for detecting and treating neovascularization.

### BACKGROUND

A major challenge for in vivo molecular imaging of pathologic mRNAs in living systems is that unmodified oligonucleotides are unstable and exhibit rapid renal clearance from circulation, leading to minimal bioavailability in target tissues. What is needed are novel compounds, compositions, and methods for detecting RNAs.

The compounds, compositions, and methods disclosed herein address these and other needs.

### SUMMARY

Disclosed herein are probes and methods for the detection of RNAs and for the detection and treatment of neovascularization. The inventors have developed a novel lipid-shRNA conjugate that facilitates delivery of anti-sense (AS) oligonucleotides into target cells without using any transfection reagents.

In some aspects, disclosed herein is a probe for the detection of an RNA, comprising: a short hairpin RNA sequence (shRNA), wherein the shRNA sequence comprises an anti-sense sequence complementary to a target sequence of the RNA; a lipid moiety conjugated to the shRNA; a quencher conjugated to the shRNA; and a fluorescent dye conjugated to the shRNA.

In some embodiments, the RNA comprises an endoglin mRNA. In some embodiments, the shRNA sequence comprises SEQ ID NO:1.

In some embodiments, the RNA comprises a HIF-1α mRNA. In some embodiments, the shRNA sequence comprises SEQ ID NO:2.

In some embodiments, the shRNA sequence comprises about 15-45 nucleotides. In some embodiments, the target sequence of the RNA is about 21 nucleotides.

In some embodiments, the shRNA comprises at least one chemically modified nucleotide. In some embodiments, the at least one chemically modified nucleotide comprises 2′-O-methyl (2′ MeO).

In some embodiments, the lipid moiety is a diacyl lipid moiety. In some embodiments, the lipid moiety is conjugated to the shRNA by a linker. In some embodiments, the lipid moiety is conjugated to the shRNA by a polyethylene glycol (PEG) linker.

In some embodiments, the quencher is BHQ-2. In some embodiments, the fluorescent dye is cyanine-3 (Cy3).

In some aspects, disclosed herein is a probe for the detection of a nucleic acid, comprising:
a short hairpin RNA sequence (shRNA), wherein the shRNA sequence comprises an anti-sense sequence complementary to a target sequence of the nucleic acid;
a lipid moiety conjugated to the shRNA;
a quencher conjugated to the shRNA; and
a fluorescent dye conjugated to the shRNA.

In some aspects, disclosed herein is a method for detecting an RNA, comprising: introducing a probe into a cell or a tissue, wherein the probe comprises;
a short hairpin RNA sequence (shRNA), wherein the shRNA sequence comprises an anti-sense sequence complementary to a target sequence of the RNA;
a lipid moiety conjugated to the shRNA;
a quencher conjugated to the shRNA; and
a fluorescent dye conjugated to the shRNA;
allowing the probe to bind the target sequence; and
detecting the fluorescent dye after the probe binds to the target sequence.

In some embodiments, the cell or tissue is an ocular cell or tissue.

In some aspects, disclosed herein is a method for detecting neovascularization, comprising:
introducing a probe into a cell or a tissue, wherein the probe comprises;
   a short hairpin RNA sequence (shRNA), wherein the shRNA sequence comprises an anti-sense sequence complementary to a target sequence of an RNA;
   a lipid moiety conjugated to the shRNA;
   a quencher conjugated to the shRNA; and
   a fluorescent dye conjugated to the shRNA;
allowing the probe to bind the target sequence; and
detecting the fluorescent dye after the probe binds to the target sequence.

In some embodiments, the cell or tissue is an ocular cell or tissue.

In some aspects, disclosed herein is a method for treating neovascularization, comprising:
detecting neovascularization in a subject, comprising:
   introducing a probe into a cell or a tissue, wherein the probe comprises;
      a short hairpin RNA sequence (shRNA), wherein the shRNA sequence comprises an anti-sense sequence complementary to a target sequence of the RNA;
      a lipid moiety conjugated to the shRNA;
      a quencher conjugated to the shRNA; and
      a fluorescent dye conjugated to the shRNA;
   allowing the probe to bind the target sequence; and
   detecting the fluorescent dye after the probe binds to the target sequence; and
      administering to the subject an effective amount of an inhibitor of neovascularization, if neovascularization is detected in the subject.

In some embodiments, the inhibitor of neovascularization is selected from bevacizumab or ranibizumab.

### BRIEF DESCRIPTION OF THE DRAWINGS

The accompanying figures, which are incorporated in and constitute a part of this specification, illustrate several aspects described below.

FIGS. 1A-1F show design and characterization of anti-sense lipid-shRNA conjugates (AS-shRNA-lipid). FIG. 1A shows schematic drawing and hybridization motif of lipid-shRNA conjugates showing the design incorporating anti-sense sequence complementary to the endoglin mRNA and are stabilized using 2′-OMe nucleotides and a fluorescence dye quenched by BHQ2 as shown. The shRNA sequence in FIG. 1A is mGmCmAmGmCmUmCmUmGmUmCmUmUmUmCmUmUmUmGmGmUmCmUmGmC mGmCmUmGmC (SEQ ID NO:11). The complementary sequence is 5′-GCAGACCAAAGAAAGACAGA-3′ (SEQ ID NO:19). The single mismatch sequence is 5′-GCAGACCGAAGAAAGACAGA-3′ (SEQ ID NO:20). Upon hybridization to the target sequence, quenching is abolished, and fluorescence occurs reporting the hybridization event. FIG. 1B shows transmission electron microscopy (TEM) images of the synthesized shRNA-lipid conjugates. FIGS. 1C-1D show that both shRNA and lipid-shRNA conjugates are highly specific for their complementary sequences and in some sequence with a single-mismatch (SMM) do not hybridize. FIGS. 1E-1F show that the lipid-shRNA binding kinetics to the complementary sequence in presence and absence of albumin showing that interaction with albumin on the terminal lipid does not affect target sequence binding.

FIGS. 2A-2H show single cell RNAseq data. Evaluation of single cell RNAseq data of the white blood cells isolated from whole blood from P17 mouse OIR and RA control mice as shown in FIGS. 2A and 2B. FIG. 2C shows representative aggregated t-SNE plot created using cellRanger-atac-aggr program showing the total UMI counts for each cell-barcode from two sample types, OIR and RA control (total 19,994 cells; 8,870 cells from OIR and 11,124 cells from RA control mice). Cells with greater UMI counts likely have higher RNA content than cells with fewer UMI counts. FIG. 2D shows representative t-SNE plot of each cell-barcode clustered by an automated clustering algorithm, clustering group of cells with similar expression profiles. In this space, pairs of cells that are close to each other have more similar gene expression profiles than cells that are distant from each other. FIG. 2E shows heatmap representation of up-regulated gene expression profiles in OIR compared to RA control animals FIG. 2F shows t-SNE plot created using Loupe Cell Browser program, shows a distinct cluster of cells (cluster 9) appeared in OIR animals (as shown by the black arrow) which is minimally present in RA control animals. FIG. 2G shows ENG gene expression profile in OIR and in RA control animals ENG is highly up-regulated in cells from OIR in cluster 9 and also some of these cells are positive for macrophage mannose receptor C-1 (MRC1) gene as shown in FIG. 2H. A minimal number of cells is observed in RA control mice in cluster 9 and were minimally positive for MRC1 gene.

FIGS. 3A-3F show that fluorescence in situ hybridization (FISH) imaging was used to visualize the distribution and intensity of endoglin (ENG) mRNA in transverse retinal section from P17 oxygen treated OIR animals and endoglin (CD105) protein in F4/80 cells in flat mount P17 OIR retinas. Endoglin was expressed in all endothelial cells and also in activated microglial cells. C57BL/6 OIR pups were treated with high oxygen (75%) from postnatal day 7 (P7) to P12 and retinal tissues were harvested for ex vivo analysis of endoglin mRNA distribution using fluorescence in situ hybridization (FISH) method using confocal microscopy. FIGS. 3A-3B show that strong endoglin mRNA-dependent fluorescence (red punctate) was localized to cells in neovascular lesions and also in all endothelial cells (EC) including choroidal endothelial cells in retinal cross-sections from OIR mice. Strong fluorescence correlating with endoglin mRNA (red) was localized to different cells in neovascular tufts. In addition, endoglin mRNA expression was observed in deep capillaries as well as in choroidal endothelial cells. FIGS. 3C-3F show that retinal vasculatures were visualized using isolectin B4 and activated microglial cells were visualized using macrophage specific marker F4/80. Endoglin (CD105) was co-localized with the macrophages and all the endothelial cells in the retina. Endoglin mRNA expression was not detectable in the cells in inner and outer nuclear layers. Abbreviations: INL=inner nuclear layer, GCL=ganglion cell layer, SCP=superficial capillary plexus, MCP=middle capillary plexus, DCP=deep capillary plexus. Scale bar 200 μm.

FIGS. 4A-4H show live imaging of endogenous mRNA in the mouse OIR retina using AS-shRNA-lipid conjugates. FIG. 4A shows schematic drawing representing specificity of shRNA-lipid delivery to the target cells in vivo in neovascularization. FIGS. 4B-4C show strong fluorescence emission correlating with AS-shRNA-lipid (red punctate) clearly visualized the probe delivery to the target cell population. FIGS. 4D-4E show that AS-shRNA-lipid (red punctate) were also localized on the surface of the retina, presumably localized in cells in the retina and presumably hybridized to endoglin mRNA. Upon hybridization to the target sequence, the shRNA-lipid conjugates retained around the neovascular tufts after 18 hours post-injection. FIGS. 4F-4H show that localization of AS-shRNA-lipid (red punctate) to the IBA1 positive cells was confirmed by ex vivo preparations. Sodium fluorescein (green) was used to visualize the vascular structures.

FIGS. 5A-5H show ex vivo validation of AS-shRNA-lipid fluorescence localized with IBA1 positive cells that are associated neovascular tufts in mouse P17 OIR retina. Isolectin B4 was used to visualize the retinal vasculatures. FIGS. 5A-5D show shRNA-lipids that are associated with neovascularization and not in normal vasculatures. The OIR mice received intraperitoneal injections of AS-ENG-shRNA-lipid conjugates. Eighteen hours post-injection, retinal tissues were analyzed ex vivo. AS-ENG-shRNA-lipid fluorescence was localized in IBA1 positive cells (arrows), indicating that endoglin and IBA1 positive activated microglia/macrophages are associated with neovascularization. FIGS. 5E-5H show that shRNA-lipids are associated with IBA1 positive cells in neovascularization in the superficial capillary plexus. Strong fluorescence emission presumably due to hybridization with endoglin mRNA in these IBA1 positive cells localized around neovascularization, showing the probe delivery to the neovascular tufts Minimal lipid-shRNAs fluorescence was observed in the normal endothelial cells that are also positive for endoglin mRNA, indicating that the probe hybridization can occur at the site away from these microvascular endothelial cells and migrated to the site of neovascularization.

FIGS. 6A-6L show that the AS-shRNA-lipid conjugates have no effect on retinal microglial activation and were minimally detectable in IBA1 positive ramified/resting microglia in room air (RA) raised healthy control retina. Isolectin B4 was used to visualize the retinal vasculatures. Microglia were observed on the surface of superficial capillary plexus (SCP), middle capillary plexus (MCP) and deep capillary plexus (DCP) and are ramified. AS-shRNA-lipid conjugates were not detectable in SCP (FIGS. 6A-6D); where most of the activated microglia/macrophages were localized in OIR retina; and were not detectable in MCP or DCP (FIGS. 6E-6L). Scale bar 20 tim.

FIGS. 7A-7E show in vivo bio-distribution, pharmacokinetics and toxicity of AS-ENG-shRNA-lipid conjugates. FIGS. 7A-7C show ex vivo imaging of the isolated organs from AS-shRNA-lipid injected animals showed that these probes have prolong bioavailability in vivo for tissue uptake and are cleared through renal secretion after 4 hours. The shRNA-lipid conjugates can deliver to the lymph nodes as shown in FIG. 7B, where they can be internalized into the macrophages and migrated to the retina. FIG. 7D shows plasma concentration time profile of shRNA-lipid showing its clearance after one hour. FIG. 7E shows cellular uptake and in vitro toxicity of AS-lipid-shRNA in retinal microvascular endothelial cells were assessed by the live-dead assay using Calcein AM. Lipid-shRNA did not significantly reduce MRMEC viability at 0.1 and 0.5 nmol concentrations compared to normal serum treated cells.

FIGS. 8A-8B show size distribution and polydispersity index measurement of the shRNA-lipid conjugate in PBS using dynamic light scattering (DLS) and molecular weight measurement using ESI-TOF. In FIG. 8A, DLS measurements showed that, sample contains one major population by volume indicating good measurement quality. Polydispersity index (PDI) value of 0.203 demonstrates a broad size range within the population indicating the presence of multiple species/nanoparticles. In FIG. 8B, the ESI-TOF MS data for shRNA-lipid conjugates at around 15 kDa also showed a series of MS from multiple shRNA-lipid conjugates with a series of PEG lengths indicate multiple species of the conjugates that might contribute to the polydispersity.

FIGS. 9A-9D show intracellular delivery of AS-shRNA-lipid conjugate into bone marrow derived macrophages (MMa-bm). Endoglin mRNA was induced in MMa-bm cells using phorbol myristate acetate (PMA) following a known protocol. FIGS. 9A-9B show that intracellular delivery of shRNA-lipids was observed in MMa-bm phagocytic macrophages and could be induced by using poly-L-lysine (PLL). FIGS. 9C-9D show that AS-shRNA-lipid derived fluorescence was minimally detected in MMa-bm without PMA treatment, indicating lower expression of endoglin mRNA.

FIGS. 10A-10D show sensitivity and specificity of AS-ENG-shRNA-lipid conjugates. Non-overlapping sequences targeting ENG mRNA were selected using RNA secondary structure prediction program MFOLD and then narrowing the best selected sequence using a second computer program, OLIGOWALK which identifies probe sequence that binds most stably to their complementary sequence. A nonsense-shRNA-lipid was designed by selecting random sequence and BLAST searched to avoid nonspecific binding. Complementary sequence designed for ENG seq-1 was used monitor off-target of the nonsense-shRNA-lipid. FIGS. 10A-10C show that the 2′-MeO nucleotides protected AS-shRNA-lipid conjugates were highly specific for their complementary sequences and were stable in presence of nonsense complementary sequence (NS-comp). FIG. 10D shows that, in this probe binding hybridization kinetic experiment, nonsense-shRNA-lipid also shows high-level of specificity for its perfect complementary sequence and remains non-responsive to the endoglin specific sequences, indicating the low levels of nonspecific binding of the nonsense-shRNA-lipid to other mRNAs.

FIG. 11 shows comparative stability of AS ENG-shRNA-lipid and NS shRNA-lipid conjugates were monitored in serum containing medium. Probes were incubated with fetal bovine serum (FBS) or PBS for 6 hours at 37° C. Both AS ENG-shRNA-lipid and NS shRNA-lipid conjugates were stable in FBS as well as in PBS for at least 6 hours, indicating similar stability in vivo. In addition, the probes responded to their corresponding complementary sequence after 6 hours in FBS or PBS by emitting fluorescence signals, indicating their retained hairpin structures before hybridization to the complementary sequence.

FIGS. 12A-12K show MS data for oligonucleotides. FIGS. 12J-12K show MS data for the oligonucleotides without BHQ to use for biodistribution assays. The sequence in FIG. 12A is mGmCmAmGmCmUmGmCmAmAmCmUmCmAmGmUmUmCmCmAmUmCmAmUmU mAmCmGmGmGmCmUmGmC-3′ (SEQ ID NO:5), with a 5AmMC6/iCy3 modification at its 5′ end and a 3BHQ_2 modification at its 3′ end. The sequence in FIG. 12B is SEQ ID NO:1 (MI-9-2018-mENG seq-2 Cy3) mGmCmAmGmCmAmCmUmGmUmGmAmUmGmUmUmGmAmCmUmCmUmUmGmG mCmGmCmUmGmC, with a 5AmMC6/iCy3 modification at its 5′ end and a 3BHQ_2 modification at its 3′ end. The sequence in FIG. 12C is 5′-mGmCmUmCmGmUmUmUmGmAmCmCmUmUmGmCmUmUmCmCmUmGmGmAmA mAmGmAmUmCmGmAmGmC-3′ (SEQ ID NO:6), with a 5AmMC6/iCy3 modification at its 5′ end and a 3BHQ_2 modification at its 3′ end. 5′-mCmCmGmGmUmUmUmAmGmUmUmCmCmUmGmUmUmCmUmGmUmUmGmUmC mUmUmCmAmCmCmGmG-3′ (SEQ ID NO:10), with a 5AmMC6/iCy3 modification at its 5′ end and a 3BHQ_2 modification at its 3′ end. The sequence in FIG. 12E is 5′-TTC CGT AAT GAT GGA ACT GAG TTG CAT T-3′ (SEQ ID NO:12). The sequence in FIG. 12F is 5′-TTG CCA AGA GTC AAC ATC ACA GTG CTT-3′ (SEQ ID NO:13). The sequence in FIG. 12G is 5′-TTA TCT TTC CAG GAA GCA AGG TCA AAT T-3′ (SEQ ID NO:14). The sequence in FIG. 12H is 5′-TTG AAG ACA ACA GAA CAG GAA CTA ATT-3′ (SEQ ID NO:15). The sequence in FIG. 12I is 5′-TTG AAG ACA ACA GAA GAG GAA CTA ATT-3′ (SEQ ID NO: 16). The sequence in FIG. 12J is 5′-mGmCmAmGmCmUmGmCmAmAmCmUmCmAmGmUmUmCmCmAmUmCmAmUmU mAmCmGmGmGmCmUmGmC-3′ (SEQ ID NO: 17), with ThioMC6-D//iCy5/modification at its 5′ end. The sequence in FIG. 12K is 5′-mCmCmGmGmUmUmUmAmGmUmUmCmCmUmGmUmUmCmUmGmUmUmGmUmC mUmUmCmAmCmCmGmG-3′ (SEQ ID NO:10), with 5ThioMC6-D//iCy5 modification at 5′-end.

FIGS. 13A-13B show HPLC and MS data for the purified compounds. FIG. 13A shows HPLC data for the purified compounds. FIG. 13B shows ESI-TOF data for shRNA-lipid conjugates. FIG. 13B shows MS around 15.1 kDa with several PEG lengths as differentiated by MS-difference of 44 Da.

FIG. 14 shows schematic drawing and hybridization motif of shRNA-lipid conjugates showing the design incorporating anti-sense sequence complementary to the HIF-1α mRNA. The shRNA-lipid conjugates are stabilized using 2′-OMe nucleotides. A fluorescence dye (FL Dye) and a black hole quencher (BHQ) are incorporated onto opposite ends of the shRNA construct as shown. Upon hybridization to the target sequence, the intramolecular quenching is compromised, and fluorescence occurs, thus reporting the hybridization event. Since the shRNA-lipid conjugates contain a hydrophilic and a lipophilic component, it is likely that they form lipid-micelle structures. From the dynamic light scattering (DLS) measurements and transmission electron microscopy (TEM) imaging, it was observed that shRNA-lipids from spherical nanoparticles of around 10 nm. The mRNA recognition moiety resides on the surface of the nanoparticles and the lipid-core remains inside. The sequence in FIG. 14 is 5′-mCmCmGmGmUmAmUmUmGmUmCmCmUmUmCmGmUmCmUmCmUmGmUmUmU mUmUmGmAmCmCmGmG-3′ (SEQ ID NO:2). The specific sequence present in the target is 5′-CAA AAA CAG AGA CGA AGG ACA AT-3′ (SEQ ID NO:18).

FIGS. 15A-15E show in vitro localization of HIF-1α mRNA targeted AS-shRNA-lipid in mouse Müller cells (MMC) using fluorescence microscopy. Expression of HIF-1α mRNA was induced in MMCs by exposing the Cells under hypoxia. DAPI was used to counterstain the nucleolus (blue). FIGS. 15A-15D show AS-shRNA-lipid derived fluorescence was minimally observed in normoxic MMCs and the fluorescence was significantly increased in hypoxic MMCs, supporting the expression of HIF-1α mRNA in hypoxic MMCs. FIG. 15E shows comparative assessment of fluorescence intensities in normoxic and hypoxic MMCs as well as in mouse retinal microvascular endothelial cells (MRMECs). Average fluorescence intensities were measured computationally using ImageJ software (n=6). Interestingly, AS-shRNA-lipid derived fluorescence was observed both in normoxic as well as in hypoxic MRMECs, suggesting that HIF-1α mRNA can be expressed constitutively in cultured MRMECs. These observations are consistent with previously observed results in other endothelial cells. Statistical significance ***P<0.0001, NS represents statistically not significant.

FIGS. 16A-16D show characterization of CD4 positive T cells, CD19 positive B cells, MRC-1 positive macrophages and expression of HIF-1α using single cell RNAseq data analysis in isolated white blood cells from P17 mouse OIR and RA control mice. FIG. 16A-16C Representative aggregated t-SNE plot showing higher numbers of CD4 positive T cells in room air (RA) control group (FIG. 16A); higher numbers of CD19 positive activated B cells in oxygen-induced retinopathy (OIR) group (FIG. 16B); macrophage mannose receptor C-1 (MRC1) positive activated cells mostly in OIR group (FIG. 16C). FIG. 16D shows that HIF-1α expression was observed in T cells, B cells and MRC1 positive macrophages. t-SNE plot was created using Loupe Cell Browser program.

FIGS. 17A-17D show imaging HIF-1α mRNA selectively in activated cells in neovascularization in mouse P17 OIR retina. Isolectin B4 was used to visualize the retinal vasculatures. FIGS. 17A-17B show the shRNA-lipid conjugates incorporating anti-sense sequence complementary to HIF-1α mRNA was localized in neovascular tufts associated cells. The OIR mice received intraperitoneal injections of AS-shRNA-lipid conjugates. Eighteen hours post-injection, retinal tissues were analyzed ex vivo. AS-shRNA-lipid fluorescence was localized in cells that are associated with IBA-1 positive cells (arrows), indicating that endoglin and IBA-1 positive activated microglia/macrophages are associated with neovascularization. FIGS. 17C-17D show the expanded panel from FIGS. 17A-17B. Strong fluorescence emission presumably due to hybridization with HIF-1α mRNA in these IBA-1 positive cells localized around neovascularization, showing the probe delivery to the neovascular tufts Minimal lipid-shRNAs fluorescence was observed in the normal endothelial cells that are also positive for HIF-1α mRNA, indicating that the probe hybridization might occur at the site away from these microvascular endothelial cells and migrated to the site of neovascularization.

FIGS. 18A-18G show co-localization of AS-shRNA-lipid derived fluorescence with IBA-1 positive cells in the P17 mouse OIR retina. FIGS. 18A-18C show that isolectin B4 was used to visualize the retinal vasculatures (green); IBA-1 was used to stain the microglial cells (magenta). FIGS. 18D-18E show that AS-shRNA-lipid derived fluorescence was localized in cells that are also associated with IBA-1 positive cells (white arrows), indicating that IBA-1 positive activated microglia/macrophages (magenta color) could express HIF-1α mRNA that are also associated with neovascularization. FIGS. 18F-18G show that AS-shRNA-lipid derived fluorescence were not observed in ramified IBA-1-positive cells that resided within the deep capillary plexus of the OIR retina.

FIGS. 19A-19D show effect of inhibitors/stimulator on shRNA-lipid internalization into the primary retinal microvascular endothelial cells (MRMEC). shRNA-lipid conjugates (both AS- and NS-) were synthesized using only the fluorescence dye (Cy5) at the 5′ end without using the black hole quencher (BHQ). Thus, the probes are not quenched and are always ON. Cells were exposed to different inhibitors or stimulators for 30 min prior to addition of the shRNA-lipid conjugates. Plate based fluorescence assays and fluorescence imaging were performed two hour post-incubation to monitor probe internalization. FIGS. 19A-19B show that Poly-L-lysine induces intracellular delivery of both AS- and NS-shRNA-lipid probe into the MRMECs, independent of the sequence present in the shRNA (red punctate). FIG. 19C shows that, in addition, shRNA-lipid internalization is independent of the macropinocytosis pathways as observed from phorbol esters treatment; and inhibited in presence of clathrin-mediated endocytosis inhibitor (sucrose), suggesting the possibility of clathrin-mediated endocytosis of shRNA-lipid, *P<0.05 (n=4). However, internalization is independent of filipin-sensitive caveolae-mediated transport. FIG. 19D shows that inhibition of endocytosis (missing punctate structures) could be visualized using confocal imaging (yellow arrows). ns=statistically not significant.

FIGS. 20A-20B show in vitro specificity and sensitivity of non-sense shRNA-lipid conjugate (NS-shRNA-lipid). Primary mouse Müller cells (MMC) were treated with NS-shRNA-lipid and exposed to hypoxia and normoxia. Fluorescence microscopy was used to localize the NS-shRNA-lipid. NS-shRNA-lipid derived fluorescence was minimally observed both in normoxic (FIG. 2A), as well as in hypoxic MMCs (FIG. 20B) under same image acquisition conditions, suggesting the retained hairpin structure of the NS-shRNA-lipid avoiding nonspecific interactions. NS-shRNA was designed from scrambled sequence of the AS-shRNA and both NS-shRNA-lipid and AS-shRNA-lipid have almost same molecular weight with same GC-contents as shown in FIG. 23.

FIGS. 21A-21L show that the AS-shRNA-lipid conjugates have no effect on retinal microglial activation and were minimally detectable in IBA1 positive ramified/resting microglia (magenta color) in room air (RA) raised healthy control retina. Isolectin B4 was used to visualize the retinal vasculatures. Microglia were observed on the surface of superficial capillary plexus (SCP), middle capillary plexus (MCP) and deep capillary plexus (DCP) and are ramified. AS-shRNA-lipid conjugates were not detectable in SCP (A-D); where most of the activated microglia/macrophages were localized in OIR retina; and were not detectable in MCP or DCP (FIGS. 21E-21L). Scale bar 20 tim.

FIGS. 22A-22D show that a non-sense shRNA (NS-shRNA) was designed using scrambled sequence of the anti-sense shRNA (AS-shRNA) and synthesized to confirm the specificity of AS-shRNA-lipid conjugate as targeted imaging probe. FIGS. 22A-22C show that NS-shRNA-lipid derived fluorescence punctate (red) was minimally detected as shown is B, in IBA1 positive macrophages (magenta) as shown in FIG. 22C, indicating the high specificity of AS-shRNA-lipid for the target HIF-1α mRNA as observed in FIG. 18. Isolectin B4 (IB4) was used as counter staining of the vascular structures in the mouse OIR retina at P17. Nonsense sequence was designed computationally and BLAST searched to confirm no significant overlap with any mouse mRNA to avoid nonspecific binding interactions. Scale bar 50 tim.

FIGS. 23A-23D show MS-data for the oligonucleotides and shRNA-lipid conjugates after purification. FIG. 23C shows that AS-shRNA-lipid conjugate contains a series of PEG lengths differentiated by 44 Da mass-differences at around 15.2-16.2 kDa observed in ESI-TOF MS data. FIG. 23D shows that NS-shRNA-lipid conjugate contains a series of PEG lengths differentiated by 44 Da mass-differences at around 15.2-16.1 kDa observed in ESI-TOF MS data. The sequence in FIG. 23A is 5′-mCmCmGmGmUmAmUmUmGmUmCmCmUmUmCmGmUmCmUmCmUmGmUmUmU mUmUmGmAmCmCmGmG-3′ (SEQ ID NO:2), with 5AmMC6//iCy3 modification at its 5′ end. The sequence in FIG. 23B is 5′-mCmCmGmGmUmUmUmAmGmUmUmCmCmUmGmUmUmCmUmGmUmUmGmUmC mUmUmCmAmCmCmGmG-3′ (SEQ ID NO:10), with 5AmMC6//iCy3 modification at its 5′ end and 3BHQ_2 at its 3′ end.

FIGS. 24A-24E show in vitro localization of HIF-1α mRNA targeted AS-shRNA-lipid in mouse retinal microvascular endothelial cells (MRMECs) using fluorescence microscopy. FIG. 24A-24D show that AS-shRNA-lipid derived fluorescence was observed both in normoxic as well as in hypoxic MRMECs, indicating that HIF-1α mRNA was expressed constitutively in cultured MRMECs. DAPI was used to counterstain the nucleolus (blue). FIG. 24E shows comparative assessment of fluorescence intensities in normoxic and hypoxic MRMECs, showing similar fluorescence intensities in both treatment groups. Fluorescence intensities were measured computationally using ImageJ software (n=6). These observations are consistent with previously reported results in endothelial cells. ns represents statistically not significant.

### DETAILED DESCRIPTION

Disclosed herein are probes and methods for the detection of RNAs and for the detection and treatment of neovascularization. The inventors have developed a novel lipid-shRNA conjugate that facilitates delivery of anti-sense (AS) oligonucleotides into target cells without using any transfection reagents.

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this disclosure belongs.

The following definitions are provided for the full understanding of terms used in this specification.

### Terminology

As used herein, the article “a,” “an,” and “the” means “at least one,” unless the context in which the article is used clearly indicates otherwise.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed herein.

The term “nucleic acid” as used herein means a polymer composed of nucleotides, e.g. deoxyribonucleotides or ribonucleotides.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” denotes single- or double-stranded nucleotide multimers. Suitable oligonucleotides may be prepared by the phosphoramidite method described by Beaucage and Carruthers, Tetrahedron Lett., 22:1859-1862 (1981), or by the triester method according to Matteucci, et al., J. Am. Chem. Soc., 103:3185 (1981), both incorporated herein by reference, or by other chemical methods using either a commercial automated oligonucleotide synthesizer or VLSIPS™ technology. When oligonucleotides are referred to as “double-stranded,” it is understood by those of skill in the art that a pair of oligonucleotides exist in a hydrogen-bonded, helical array typically associated with, for example, DNA. In addition to the 100% complementary form of double-stranded oligonucleotides, the term “double-stranded,” as used herein is also meant to refer to those forms which include such structural features as bulges and loops, described more fully in such biochemistry texts as Stryer, Biochemistry, Third Ed., (1988), incorporated herein by reference for all purposes.

The term “polynucleotide” refers to a single or double stranded polymer composed of nucleotide monomers.

The term “polypeptide” refers to a compound made up of a single chain of D- or L-amino acids or a mixture of D- and L-amino acids joined by peptide bonds.

The term “complementary” refers to the topological compatibility or matching together of interacting surfaces of a probe molecule and its target. Thus, the target and its probe can be described as complementary, and furthermore, the contact surface characteristics are complementary to each other.

The term “hybridization” refers to a process of establishing a non-covalent, sequence-specific interaction between two or more complementary strands of nucleic acids into a single hybrid, which in the case of two strands is referred to as a duplex.

The term “anneal” refers to the process by which a single-stranded nucleic acid sequence pairs by hydrogen bonds to a complementary sequence, forming a double-stranded nucleic acid sequence, including the reformation (renaturation) of complementary strands that were separated by heat (thermally denatured).

The term “melting” refers to the denaturation of a double-stranded nucleic acid sequence due to high temperatures, resulting in the separation of the double strand into two single strands by breaking the hydrogen bonds between the strands.

The term “target” refers to a molecule that has an affinity for a given probe. Targets may be naturally-occurring or man-made molecules. Also, they can be employed in their unaltered state or as aggregates with other species.

The term “promoter” or “regulatory element” refers to a region or sequence determinants located upstream or downstream from the start of transcription and which are involved in recognition and binding of RNA polymerase and other proteins to initiate transcription. Promoters need not be of bacterial origin, for example, promoters derived from viruses or from other organisms can be used in the compositions, systems, or methods described herein. The term “regulatory element” is intended to include promoters, enhancers, internal ribosomal entry sites (IRES), and other expression control elements (e.g. transcription termination signals, such as polyadenylation signals and poly-U sequences). Such regulatory elements are described, for example, in Goeddel, Gene Expression Technology: Methods in Enzymology 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g. 1, 2, 3, 4, 5, or more pol I promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EFla promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981). It is appreciated by those skilled in the art that the design of the expression vector can depend on such factors as the choice of the host cell to be transformed, the level of expression desired, etc.

The term “recombinant” refers to a human manipulated nucleic acid (e.g. polynucleotide) or a copy or complement of a human manipulated nucleic acid (e.g. polynucleotide), or if in reference to a protein (i.e, a “recombinant protein”), a protein encoded by a recombinant nucleic acid (e.g. polynucleotide). In embodiments, a recombinant expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In another example, a recombinant expression cassette may comprise nucleic acids (e.g. polynucleotides) combined in such a way that the nucleic acids (e.g. polynucleotides) are extremely unlikely to be found in nature. For instance, human manipulated restriction sites or plasmid vector sequences may flank or separate the promoter from the second nucleic acid (e.g. polynucleotide). One of skill will recognize that nucleic acids (e.g. polynucleotides) can be manipulated in many ways and are not limited to the examples above.

The term “expression cassette” refers to a nucleic acid construct, which when introduced into a host cell, results in transcription and/or translation of a RNA or polypeptide, respectively. In embodiments, an expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In some embodiments, an expression cassette comprising a terminator (or termination sequence) operably linked to a second nucleic acid (e.g. polynucleotide) may include a terminator that is heterologous to the second nucleic acid (e.g. polynucleotide) as the result of human manipulation. In some embodiments, the expression cassette comprises a promoter operably linked to a second nucleic acid (e.g. polynucleotide) and a terminator operably linked to the second nucleic acid (e.g. polynucleotide) as the result of human manipulation. In some embodiments, the expression cassette comprises an endogenous promoter. In some embodiments, the expression cassette comprises an endogenous terminator. In some embodiments, the expression cassette comprises a synthetic (or non-natural) promoter. In some embodiments, the expression cassette comprises a synthetic (or non-natural) terminator.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (www.ncbi.nlm.nih.gov). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

The phrase “codon optimized” as it refers to genes or coding regions of nucleic acid molecules for the transformation of various hosts, refers to the alteration of codons in the gene or coding regions of polynucleic acid molecules to reflect the typical codon usage of a selected organism without altering the polypeptide encoded by the DNA. Such optimization includes replacing at least one, or more than one, or a significant number, of codons with one or more codons that are more frequently used in the genes of that selected organism.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, operably linked nucleic acids (e.g. enhancers and coding sequences) do not have to be contiguous Linking is accomplished by ligation at convenient restriction sites. If such sites do not exist, the synthetic oligonucleotide adaptors or linkers are used in accordance with conventional practice. In embodiments, a promoter is operably linked with a coding sequence when it is capable of affecting (e.g. modulating relative to the absence of the promoter) the expression of a protein from that coding sequence (i.e., the coding sequence is under the transcriptional control of the promoter).

The term “nucleobase” refers to the part of a nucleotide that bears the Watson/Crick base-pairing functionality. The most common naturally-occurring nucleobases, adenine (A), guanine (G), uracil (U), cytosine (C), and thymine (T) bear the hydrogen-bonding functionality that binds one nucleic acid strand to another in a sequence specific manner. As used throughout, by a “subject” (or a “host”) is meant an individual. Thus, the “subject” can include, for example, domesticated animals, such as cats, dogs, etc., livestock (e.g., cattle, horses, pigs, sheep, goats, etc.), laboratory animals (e.g., mouse, rabbit, rat, guinea pig, etc.) mammals, non-human mammals, primates, non-human primates, rodents, birds, reptiles, amphibians, fish, and any other animal. The subject can be a mammal such as a primate ora human.

The term “about” as used herein when referring to a measurable value such as an amount, a percentage, and the like, is meant to encompass variations of ±20%, ±10%, ±5%, or ±1% from the measurable value.

### Probes and Methods

In some aspects, disclosed herein is a probe for the detection of an RNA, comprising: a short hairpin RNA sequence (shRNA), wherein the shRNA sequence comprises an anti-sense

sequence complementary to a target sequence of the RNA;

a lipid moiety conjugated to the shRNA;
a quencher conjugated to the shRNA; and
a fluorescent dye conjugated to the shRNA.

In some embodiments, the RNA is an mRNA. In some embodiments, the RNA comprises an endoglin mRNA. In some embodiments, the shRNA sequence comprises SEQ ID NO:1. In some embodiments, the RNA comprises a human endoglin mRNA.

In some embodiments, the RNA comprises a HIF-1α mRNA. In some embodiments, the shRNA sequence comprises SEQ ID NO:2.

In some embodiments, the RNA comprises a human HIF-1α mRNA.

In some embodiments, the shRNA sequence comprises SEQ ID NO:1, or a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO:1. In some embodiments, the shRNA sequence comprises SEQ ID NO:2, or a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO:2.

In some embodiments, the RNA comprises an endoglin mRNA, or a fragment thereof, or a sequence at least 60% (for example, at least 60%, at least 65%, at least 70%, at least 75%, at least 80%, at least 85%, at least 90%, at least 95%, at least 96%, at least 97%, at least 98%, at least 99%) identical to the endoglin mRNA, or the fragment thereof. In some embodiments, the RNA comprises a HIF-1α mRNA, or a fragment thereof, or a sequence at least 60% (for example, at least 60%, at least 65%, at least 70%, at least 75%, at least 80%, at least 85%, at least 90%, at least 95%, at least 96%, at least 97%, at least 98%, at least 99%) identical to the HIF-1α mRNA, or the fragment thereof.

In some embodiments, the shRNA sequence comprises about 15-45 nucleotides. In some embodiments, the shRNA sequence comprises about 20-40 nucleotides. In some embodiments, the shRNA sequence comprises about 25-35 nucleotides. In some embodiments, the shRNA sequence comprises about 30-34 nucleotides. In some embodiments, the shRNA sequence comprises about 15, about 20, about 25, about 30, about 35, about 40, about 45, or more nucleotides.

In some embodiments, the target sequence of the RNA is about 21 nucleotides. In some embodiments, the target sequence of the RNA is about 10-35 nucleotides. In some embodiments, the target sequence of the RNA is about 15-30 nucleotides. In some embodiments, the target sequence of the RNA is about 18-25 nucleotides. In some embodiments, the target sequence of the RNA is about 20-24 nucleotides. In some embodiments, the target sequence of the RNA is about 15, 16, 17, 18, 19, 20, 21, 22, 23, 24, 25, 26, 27, 28, 29, 30, or more nucleotides.

In some embodiments, the shRNA comprises at least one chemically modified nucleotide. In some embodiments, the at least one chemically modified nucleotide comprises a chemically modified nucleobase, a chemically modified ribose, a chemically modified phosphodiester linkage, or a combination thereof.

In some embodiments, the at least one chemically modified nucleotide is a chemically modified ribose. In some embodiments, the chemically modified ribose is 2′-O-methyl (2′-O-Me or 2′MeO or 2′-MeO) or 2′-fluoro (2′-F). In some embodiments, the chemically modified ribose is 2′-O-methyl (2′MeO). In some embodiments, the chemically modified ribose is 2′-fluoro (2′-F).

In some embodiments, the at least one chemically modified nucleotide is a chemically modified phosphodiester linkage. In some embodiments, the chemically modified phosphodiester linkage is phosphorothioate (PS). In some embodiments, all the nucleotides comprise chemically modified phosphodiester linkages. In some embodiments, the chemically modified phosphodiester linkages are phosphorothioate (PS).

In some embodiments, the at least one chemically modified nucleotide is a locked nucleic acid (LNA). Locked nucleic acids (LNA) can be used to stabilize the probe for in vivo delivery.

In some embodiments, the lipid moiety is a diacyl lipid moiety. In some embodiments, the lipid moiety is a monoacyl lipid moiety. In some embodiments, the lipid moiety is an unsaturated lipid moiety. In some embodiments, the lipid moiety is conjugated to the shRNA by a linker. In some embodiments, the lipid moiety is conjugated to the shRNA by a polyethylene glycol (PEG) linker.

In some embodiments, the quencher is BHQ-2.

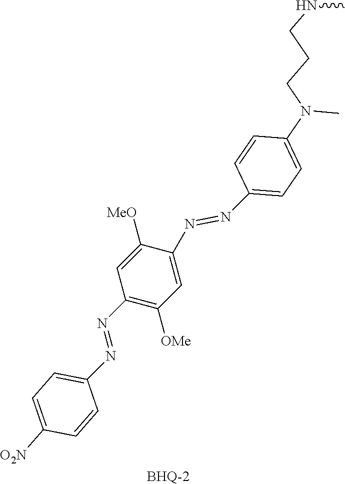

In some embodiments, the fluorescent dye is cyanine-3 (Cy3).

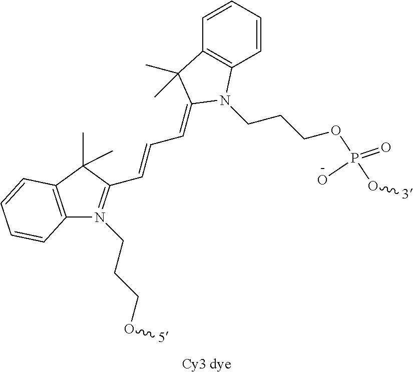

In some embodiments, the fluorescent dye is Cy5.

In some aspects, disclosed herein is a probe for the detection of an mRNA, comprising: a short hairpin RNA sequence (shRNA), wherein the shRNA sequence comprises an anti-sense

sequence complementary to a target sequence of the mRNA;
a lipid moiety conjugated to the shRNA;
a quencher conjugated to the shRNA; and
a fluorescent dye conjugated to the shRNA.

In some aspects, disclosed herein is a probe for the detection of a nucleic acid, comprising:
a short hairpin RNA sequence (shRNA), wherein the shRNA sequence comprises an anti-sense sequence complementary to a target sequence of the nucleic acid;
a lipid moiety conjugated to the shRNA;
a quencher conjugated to the shRNA; and
a fluorescent dye conjugated to the shRNA.

In some aspects, disclosed herein is a method for detecting an RNA, comprising: introducing a probe into a cell or a tissue, wherein the probe comprises;
a short hairpin RNA sequence (shRNA), wherein the shRNA sequence comprises an anti-sense sequence complementary to a target sequence of the RNA;
a lipid moiety conjugated to the shRNA;
a quencher conjugated to the shRNA; and
a fluorescent dye conjugated to the shRNA;
allowing the probe to bind the target sequence; and
detecting the fluorescent dye after the probe binds to the target sequence.

In some embodiments, the cell or tissue is an ocular cell or tissue.

In some aspects, disclosed herein is a method for detecting neovascularization, comprising:
introducing a probe into a cell or a tissue, wherein the probe comprises;
   a short hairpin RNA sequence (shRNA), wherein the shRNA sequence comprises an anti-sense sequence complementary to a target sequence of an RNA;
   a lipid moiety conjugated to the shRNA;
   a quencher conjugated to the shRNA; and
   a fluorescent dye conjugated to the shRNA;
allowing the probe to bind the target sequence; and
detecting the fluorescent dye after the probe binds to the target sequence.

In some embodiments, the cell or tissue is an ocular cell or tissue.

In some aspects, disclosed herein is a method for treating neovascularization, comprising:
detecting neovascularization in a subject, comprising:
   introducing a probe into a cell or a tissue, wherein the probe comprises;
      a short hairpin RNA sequence (shRNA), wherein the shRNA sequence comprises an anti-sense sequence complementary to a target sequence of the RNA;
      a lipid moiety conjugated to the shRNA;
      a quencher conjugated to the shRNA; and
      a fluorescent dye conjugated to the shRNA;
   allowing the probe to bind the target sequence; and
   detecting the fluorescent dye after the probe binds to the target sequence; and
      administering to the subject an effective amount of an inhibitor of neovascularization, if neovascularization is detected in the subject.

In some embodiments, the detection of the fluorescent dye is compared to a control (for example, a control sample, or a control probe). In some embodiments, the increased fluorescence (as compared to a control) indicates neovascularization. In some embodiments, the increased fluorescence (as compared to a control) indicates detection of the nucleic acid (for example, an RNA).

In some embodiments, the inhibitor of neovascularization is selected from bevacizumab or ranibizumab. Bevacizumab (trade name Avastin), is a medication used to treat neovascularization in a number of proliferative eye diseases. Ranibizumab (trade name Lucentis) is a monoclonal antibody fragment (Fah) that has been approved to treat “wet” form of age-related macular degeneration (wet AMD), a common form of age-related vision threatening eye condition.

In some embodiments, the nucleic acids herein are recombinant. In some embodiments, the nucleic acids herein are isolated. In some embodiments, the probes herein are recombinant. In some embodiments, the probes herein are isolated.

The probes herein are used for the development of a non-invasive method for detecting, measuring and imaging pathologic mRNA biomarkers including but not limited to VEGF, endoglin, HIF-1α, VCAM-1, ICAM-1, VEGFR2, IL-1b, cytokines, COX-2 mRNA. While the shRNAs herein have targeted selected sequences, any other fragment sequence that can specifically bind the mRNA can also be used. The accession number for human endoglin (ENG) is mRNA: NM_001114753.2; and the accession number for human HIF-1 alpha is NM_001243084.1. Accession numbers for all genes can be found at the National Center for Biotechnology Information website (www.ncbi.nlm.nih.gov). In some embodiments, the RNA comprises a VEGF, endoglin, HIF-1α, VCAM-1, ICAM-1, VEGFR2, IL-1b, cytokines, COX-2 mRNA, or a fragment thereof, or a sequence at least 60% (for example, at least 60%, at least 65%, at least 70%, at least 75%, at least 80%, at least 85%, at least 90%, at least 95%, at least 96%, at least 97%, at least 98%, at least 99%) identical to the VEGF, endoglin, HIF-1α, VCAM-1, ICAM-1, VEGFR2, IL-1b, cytokines, COX-2 mRNA, or the fragment thereof. These methods are used for retinal imaging of mRNA biomarkers in a retinal vascular disease.

Thus, in some aspects, disclosed herein is a method for detecting a retinal vascular disease, comprising:
introducing a probe into a cell or a tissue, wherein the probe comprises;
   a short hairpin RNA sequence (shRNA), wherein the shRNA sequence comprises an anti-sense sequence complementary to a target sequence of an RNA;
   a lipid moiety conjugated to the shRNA;
   a quencher conjugated to the shRNA; and
   a fluorescent dye conjugated to the shRNA;
allowing the probe to bind the target sequence; and
detecting the fluorescent dye after the probe binds to the target sequence.

In some aspects, disclosed herein is a method for treating a retinal vascular disease, comprising:
detecting neovascularization in a subject, comprising:
   introducing a probe into a cell or a tissue, wherein the probe comprises;
      a short hairpin RNA sequence (shRNA), wherein the shRNA sequence comprises an anti-sense sequence complementary to a target sequence of an RNA;
      a lipid moiety conjugated to the shRNA;
      a quencher conjugated to the shRNA; and
      a fluorescent dye conjugated to the shRNA;
   allowing the probe to bind the target sequence; and
   detecting the fluorescent dye after the probe binds to the target sequence; and
      administering to the subject an effective amount of a therapeutic agent to treat the retinal vascular disease, if the target sequence of the RNA is detected in the subject.

In some embodiments, the RNA is an mRNA. In some embodiments, the cell or tissue is an ocular cell or tissue. In some embodiments, the cell or tissue is a retinal cell or tissue.

In some embodiments, the retinal vascular disease is selected from proliferative diabetic retinopathy (PDR), age-related macular degeneration (AMD), retinopathy of prematurity (ROP), retinal vein occlusion, or ocular cancer. Ranibizumab can be used to treat macular edema caused by diabetic retinopathy. Ranibizumab can also be used to treat choroidal neovascularization in AMD. Another drug, bevacizumab (trade name Avastin), can also be used to treat AMD. Laser therapy can be used to treat advanced ROP. Cryotherapy can be used to freeze a specific part of the eye that extends beyond the edges of the retina. Ranibizumab or bevacizumab can be used to treat retinal vein occlusion. Radiation therapy, laser therapy and/or surgical resection (removal of the tumor) and/or enucleation are common treatment options for ocular cancer.

### EXAMPLES

The following examples are set forth below to illustrate the compounds, probes, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: A Novel Method to Visualize and Track a Sub-Population of Bone Marrow Derived Cells in Pathological Neovascularization

Diabetic retinopathy (DR) is a vision-threatening condition that affects a large number of diabetic patients within the working age population worldwide. The early stage, referred to as non-proliferative DR (NPDR), is partially characterized by retinal vaso-regression (ischemia) leading to hypoxia. Proliferative DR (PDR), constitutes the late stage, and it is defined by the development of pre-retinal neovascularization characterized by the formation of neovascular structures at the vitreoretinal interface. These structures or ‘neovascular tufts’ are often associated with hemorrhaging and tractional retinal detachment that may lead to blindness. Although the pathogenic mechanisms underlying PDR are largely unknown, ischemia-induced hypoxia and the release of hypoxia-dependent vascular endothelial growth factor (VEGF), in addition to other vasoactive and/or proinflammatory factors, are of central importance.

Evidence shows that circulating endothelial progenitor cells and macrophages migrate to the retina in response to neovascularization. However, the exact role of these migrating cells and macrophages to induce or promote NPDR or PDR that is associated with neovascularization is largely unknown. In response to neovascularization such as that occurring in proliferative diabetic retinopathy, microglia become activated, releasing pro-angiogenic and pro-inflammatory mediators and possibly contributing to neovascularization. However, technical difficulties are the major hurdle against characterizing this small number of activated cells in the retina. In addition, contribution from other cells to retinopathy is a possibility.

Endoglin (CD105) is a transmembrane auxiliary receptor for transforming growth factor-beta (TGF-β) that is predominantly expressed in proliferating vascular endothelial cells, and bone marrow-derived endothelial progenitor cells. Very little is known about the role of endoglin in human PDR, though soluble endoglin (sEng) levels are increased in the vitreous and blood of PDR patients and in the retinas of experimental models of diabetes. It is speculated that sEng is a proteolytic cleavage product of the full-length protein. The shRNA knockdown and use of neutralizing antibodies against endoglin in cell-based assays that model angiogenic components of PDR, indicated that endoglin has proangiogenic function. Endoglin (CD105) protein is associated with neovascularization. In line with these observations, real-time imaging of endoglin mRNA that correlates with the onset, progression and resolution can possibly predicts the risk of neovascularization. Fluorescence in situ hybridization (FISH) is a powerful method to visualize intracellular mRNA localization in ex vivo tissue preparations and is capable of distinguishing RNA molecules that differ in only a single base. Other hybridization methods include the use of molecular beacon and forced intercalation probes (FIT). Additional methods to visualize mRNA include covalent modification of mRNA, mRNA binding proteins, and reporter protein expression by trans-splicing to visualize mRNA. However, most of these hybridization methods require the use of fixed tissues or endogenously labelled target mRNA for imaging and tracking. Recent development of gold-mediated targeted delivery of oligonucleotides facilitates the real-time imaging of mRNA in living cells. In this example, AS-shRNA-lipid conjugates were designed and synthesized for targeted imaging of endoglin mRNA that is associated with neovascularization in living retina without using any toxic transfection reagents. This technology can be used for mRNA imaging in the clinic to monitor disease onset, pathologic progression and response to therapy.

### Results

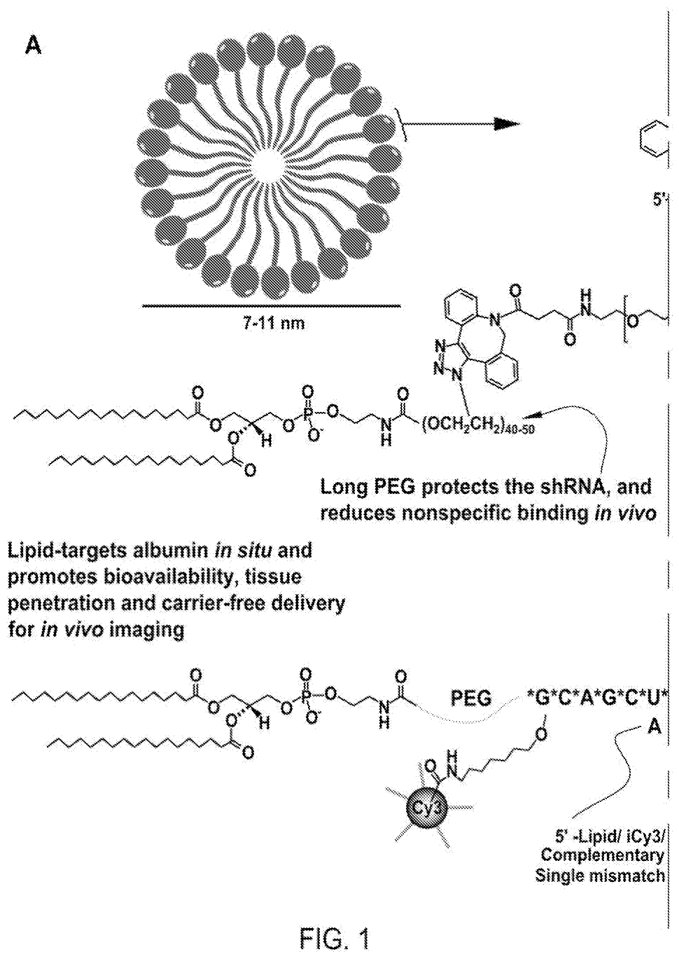

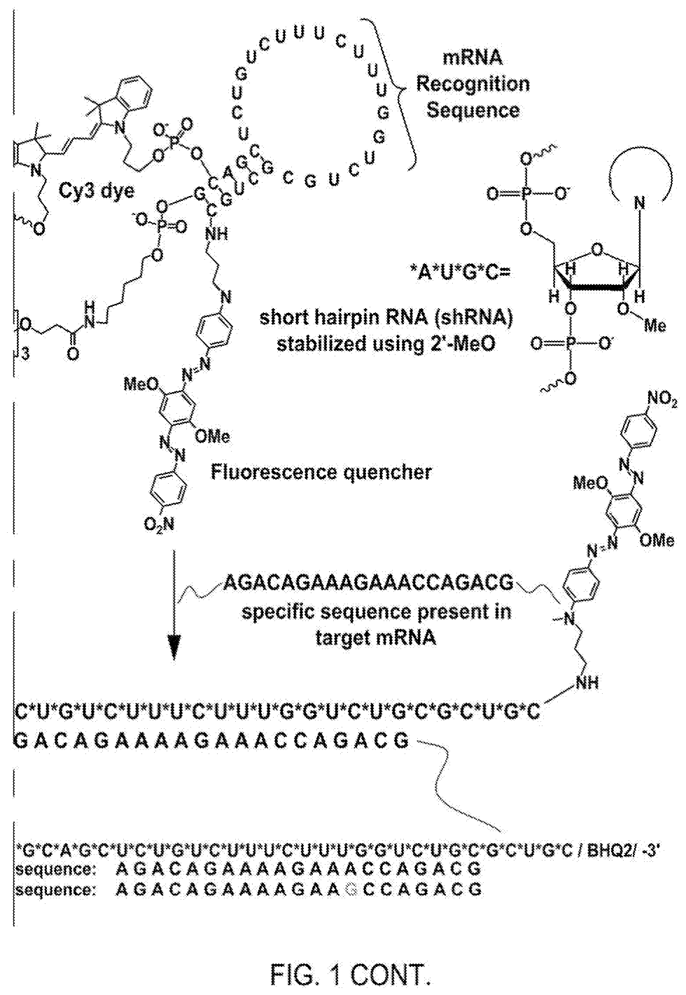

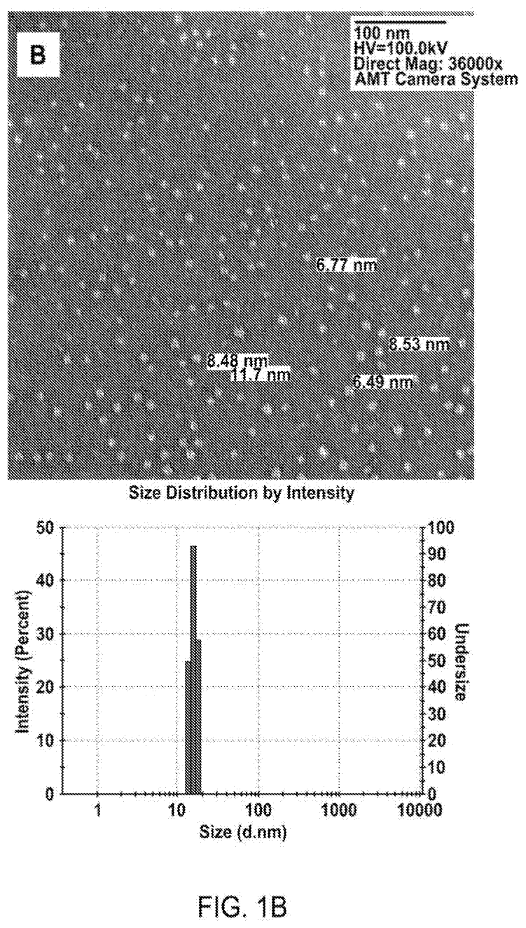

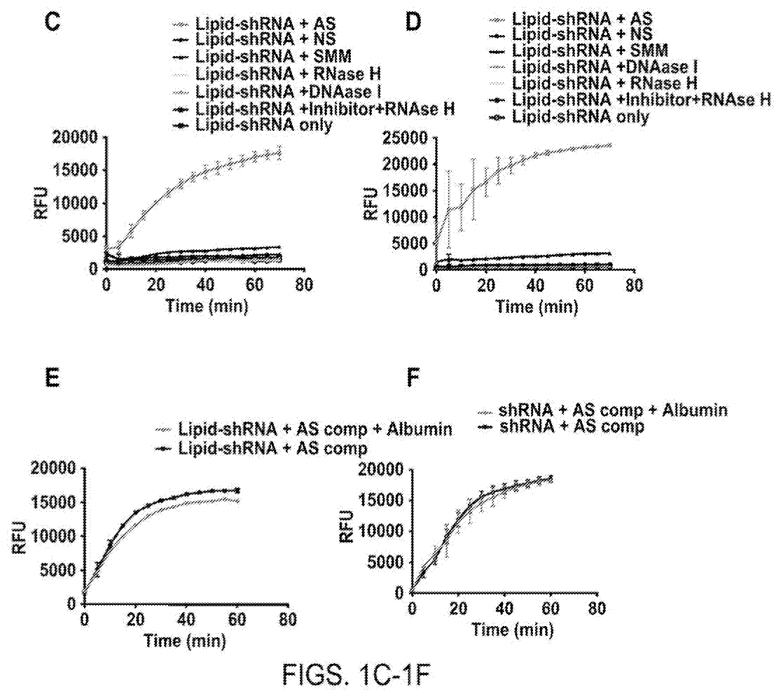

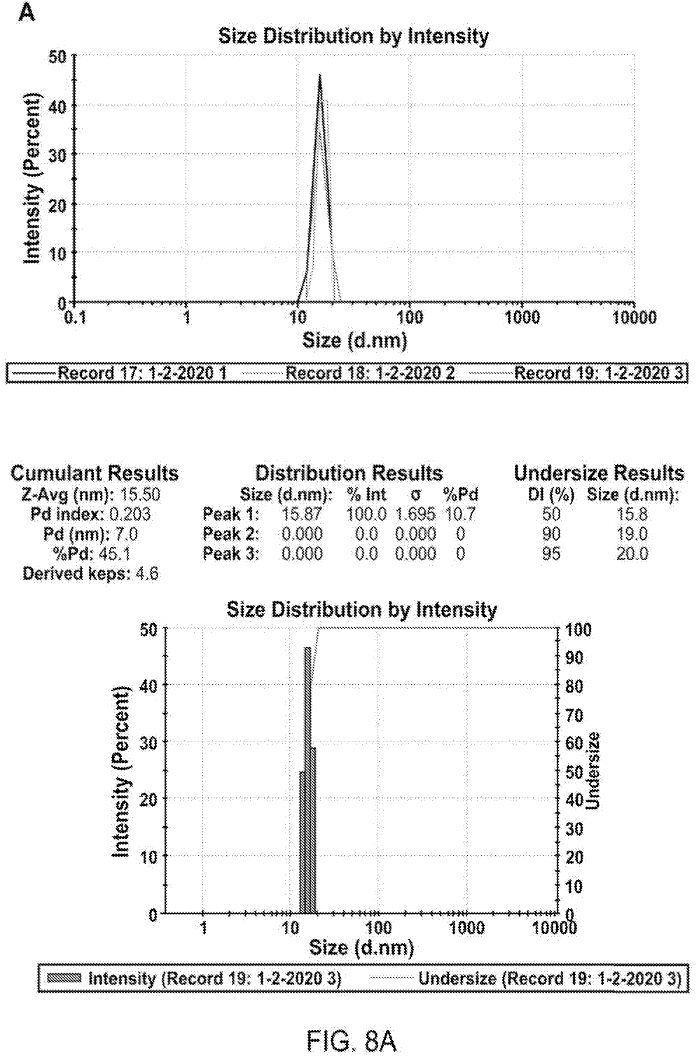

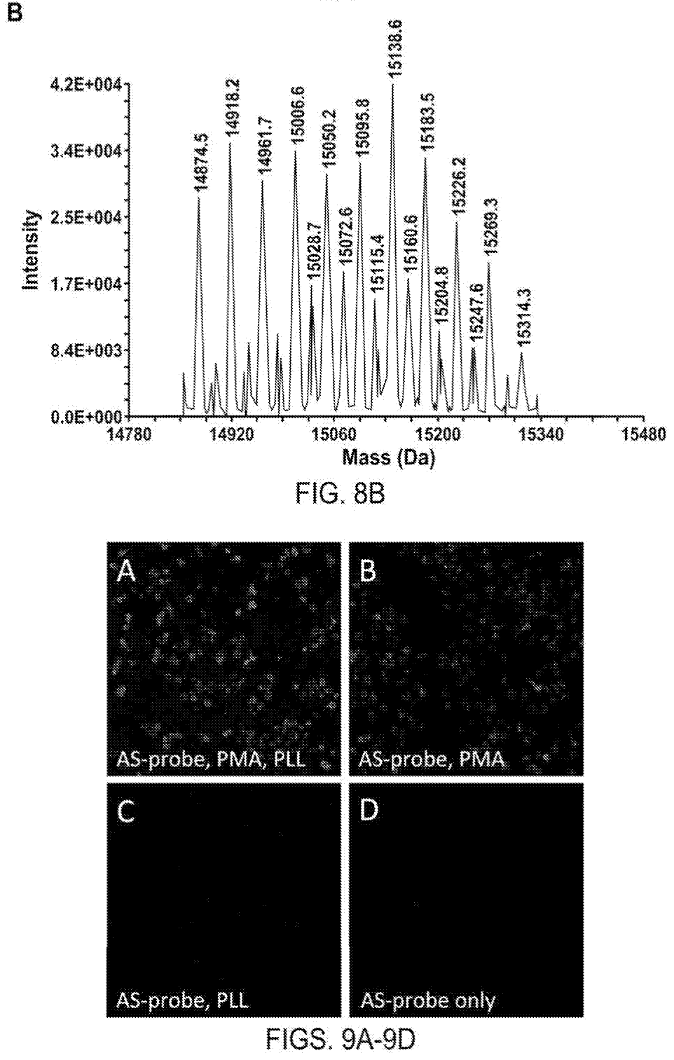

Design and synthesis of shRNA-lipid conjugates. In order for the probe design, computational analysis of the shRNA was used to target endoglin mRNA with high specificity. The region in endoglin mRNA was selected on the basis of accessibility of shRNA as predicted by RNA secondary structure predictions using MFOLD software. Then, the best candidate sequence was determined using the OLIGOWALK software based on the probe sequence predicted to bind most stably to its complementary sequence. After designing and selecting the best sequence, nuclease-resistant shRNA was synthesized with 2′-O-methylribonucleotide (2′-OMe) modified RNA chemistry. A fluorescence dye introduced at the 5′ end was quenched by black-hole quencher-2 (BHQ2) introduced at the 3′ end of the oligonucleotide. The AS(or NS)-shRNA products were purified using high-pressure liquid chromatography through a C-18 reverse-phase column. A diacyl-lipid was then conjugated in two steps using a previously developed method as described in order for transfection agent-free delivery of shRNA to the neovascular lesions. Physical properties of shRNA-lipid conjugates were monitored using transmission electron microscopy (TEM) and dynamic light scattering (DLS) (FIG. 1). Since the shRNA-lipid conjugates contain hydrophilic and lipophilic components, it is likely that they can form lipid-micelle structures. From DLS measurements it was observed that in solution shRNA-lipid conjugates contained one major population of nanoparticles by volume, indicating good measurement quality. However, polydispersity index value of 0.203 demonstrates a broad size range within the population indicating the presence of multiple species/nanoparticles. This multi-species nanoformulation is due to shRNA-lipid conjugates with a series of PEG lengths as observed in ESI-TOF MS data of the shRNA-lipid conjugates at around 15 kDa as shown in FIGS. 8A-8B contributing to high polydispersity index.

Signal-to-background ratios were measured from hybridization kinetics in the presence of the target sequence (FIG. 1). Upon hybridization to a complementary oligonucleotide sequence present in the target mRNA, the fluorophore is de-quenched, allowing strong fluorescence emission by a factor of several thousand with sensitivity enhancement of about 100-fold as shown in FIGS. 1C and 1D and FIG. 9. The target sequence detection is highly specific and demonstrated the capacity to discriminate a single mismatch in the target mRNA. To test the nuclease sensitivity, AS-shRNA-lipid conjugates were treated with DNase I and subsequent changes in fluorescence were measured as function of time. Modification with 2′-O-methylribonucleotides protects the AS-shRNA-lipid from DNase I mediated degradation. Analysis of shRNA-lipid binding kinetics with complementary sequence in presence or absence of albumin showed that the shRNA-lipid bound with complementary mRNA recognition sequences regardless of the presence of albumin (FIGS. 1E-1F).

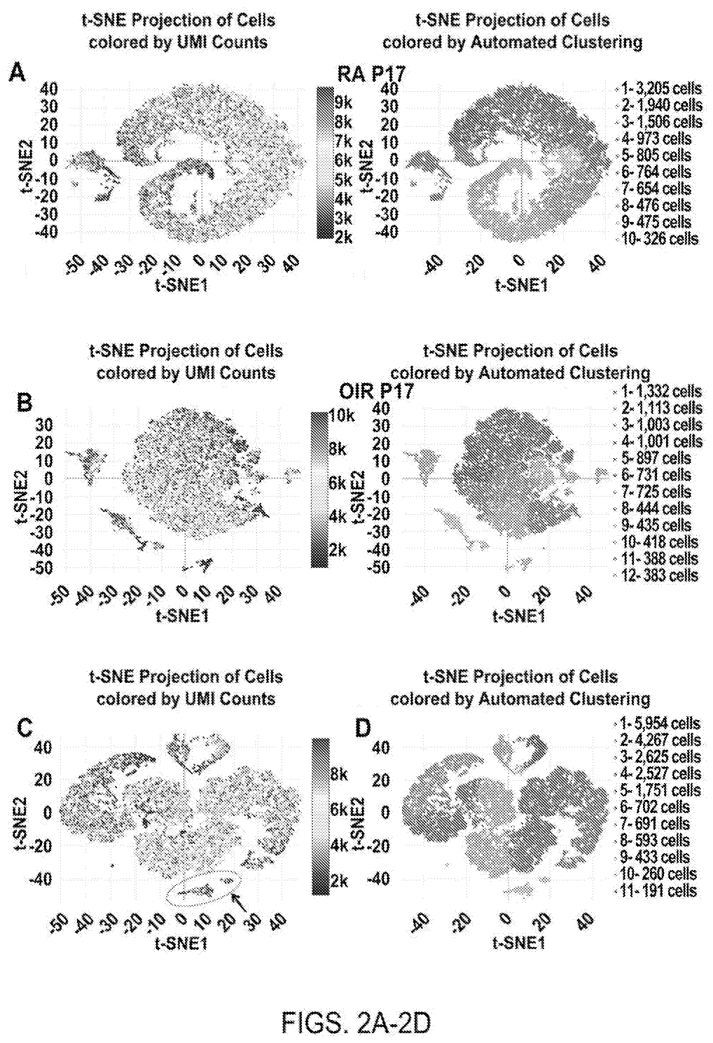

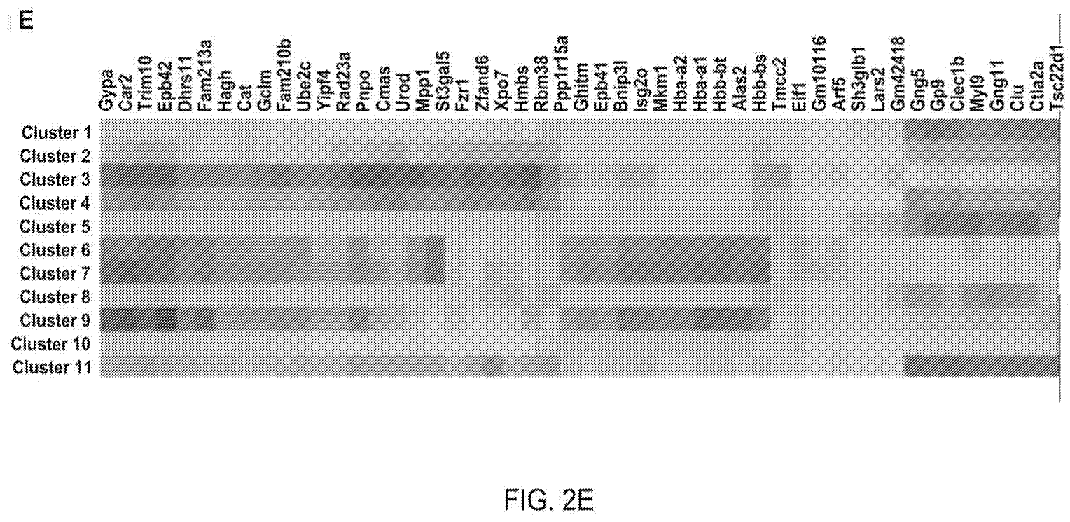

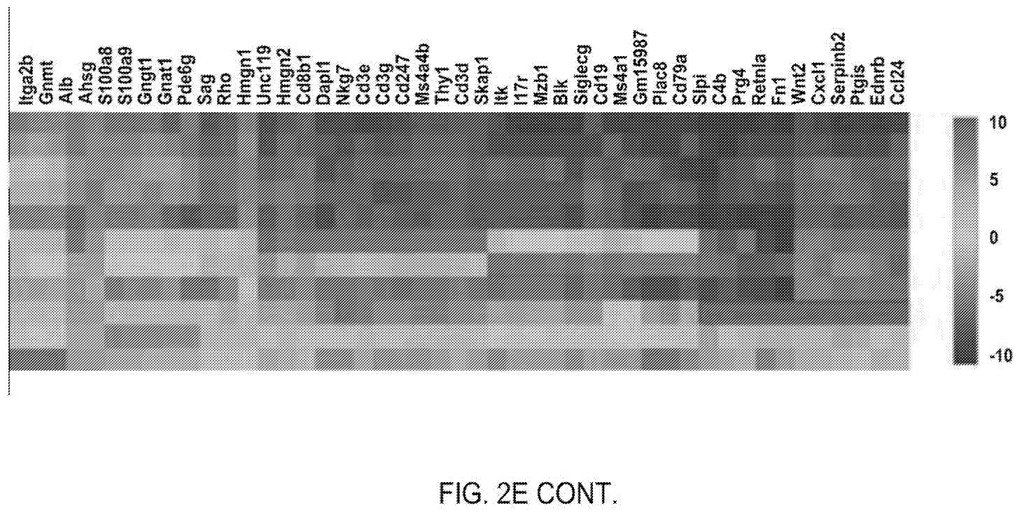

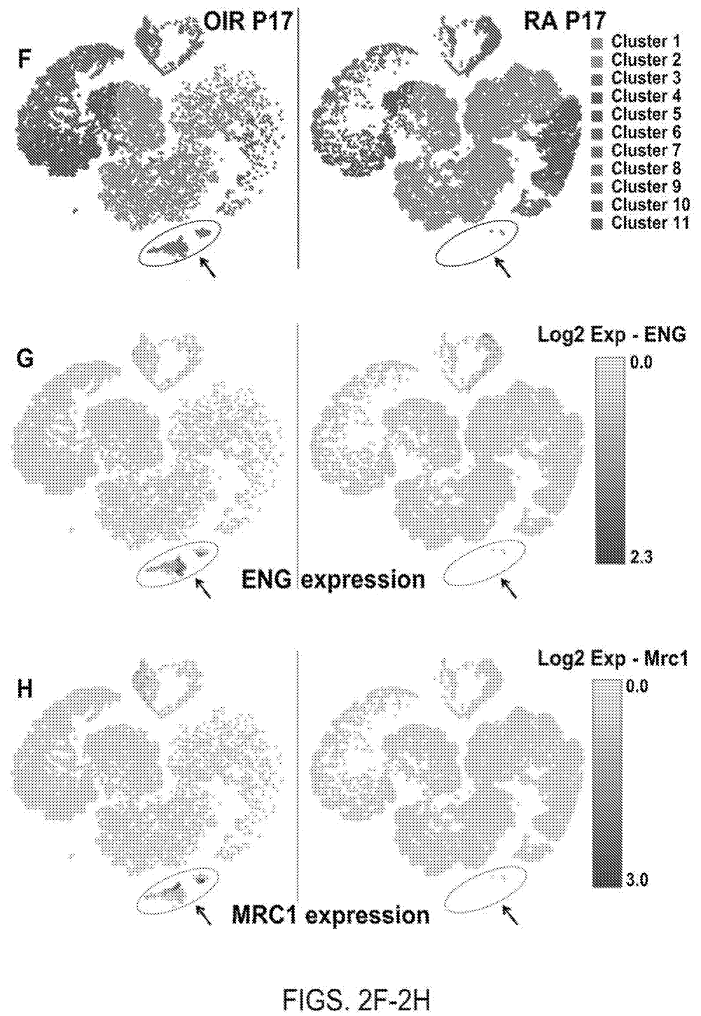

Single-cell profiling of endoglin (ENG)-positive bone marrow-derived cells in peripheral blood from mouse oxygen-induced retinopathy (OIR). To determine a specific population of bone marrow-derived cells in mouse OIR, an experimental and a computational strategy were developed to identify a cluster of cells that are distinct in OIR mouse and minimally present in room air (RA) controls (FIG. 2). Cells having a very small population (<1%) can escape from detection in a population-based assay that includes many cell types in the retina. For example, photoreceptors number over 120,000,000, while resident microglia are estimated to peak at only 10,000 when recruited to the retina under disease conditions. In addition, activated microglia resided on the surface of the superficial capillary plexus of the OIR retina, whereas resting microglia resided in the middle and deep capillary plexuses. Thus, to identify endoglin mRNA in this small population of cells isolated from OIR retina constitutes a significant challenge. To overcome some of these limitations, single-cell RNA sequencing data analysis was used to better assess the diversity of cells in peripheral blood from OIR mouse and compare with healthy controls. This analysis enabled the identification of a new subpopulation of bone marrow-derived cells from mouse OIR that are both positive for ENG and are also associated with discriminative genes, including MRC-1.

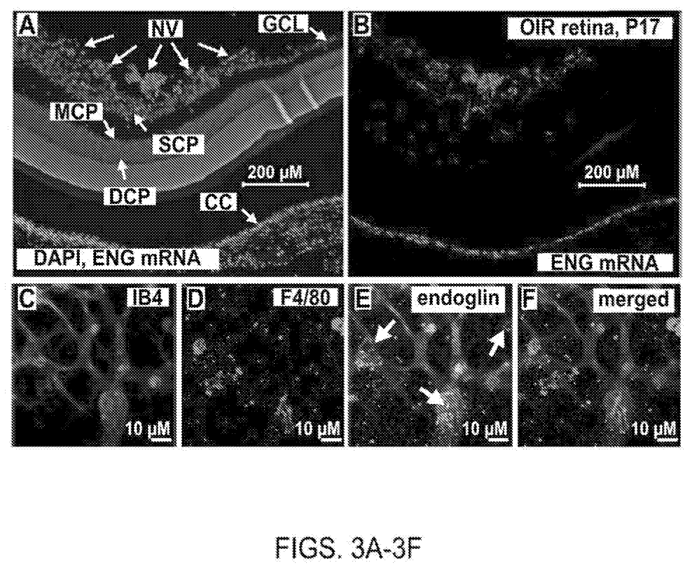

Distribution of endoglin (ENG) mRNA in OIR retina. Fluorescence in situ hybridization (FISH) was used to localize expression of endoglin mRNA in the excised OIR retina, and immunostaining was used to co-localize endoglin (CD105) in F4/80-positive cells (FIG. 3). To produce OIR, C57BL/6 pups were treated with high oxygen (75%) from postnatal day 7 (P7) to P12, and retinal tissues were harvested at P17 for ex vivo analysis of endoglin mRNA distribution using confocal microscopy. With this technique, endoglin mRNA was localized in neovascular tufts presumably in endothelial cells and also in F4/80 positive cells in the retina. In addition, endoglin mRNA was localized to the capillaries within the OIR retina and choriocapillaris.

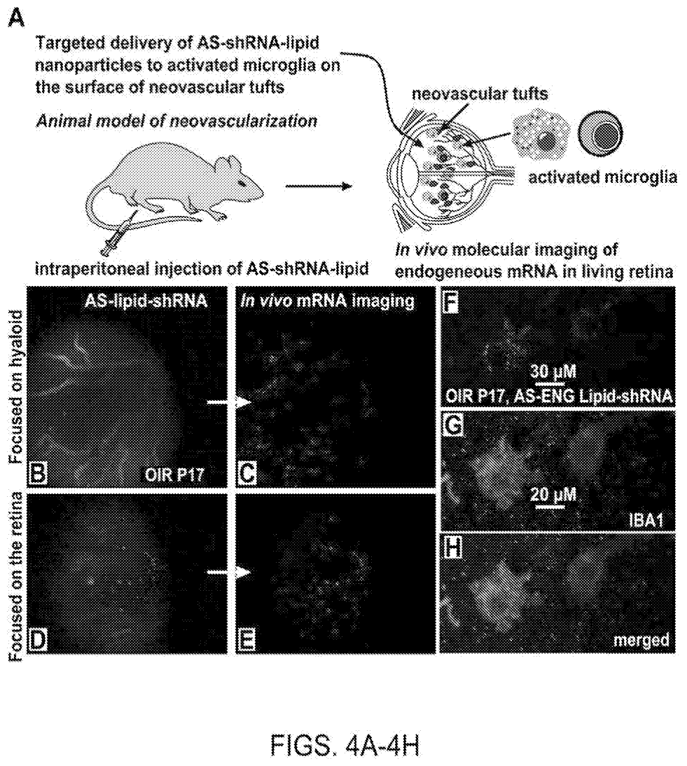

Direct imaging of endogenous mRNA in living retina using AS-shRNA-lipid. After confirming the association of endoglin mRNA with F4/80-positive cells, the molecular beacon AS-shRNA-lipid conjugate that incorporated an anti-sense sequence complementary was used to endoglin mRNA for molecular imaging of activated microglial cells in the OIR retina (FIG. 4). After intraperitoneal injection in OIR animals, it was observed that AS-shRNA-lipid yielded a strong punctate fluorescence in cells that were also positive for ionized calcium-binding adaptor molecule 1 (IBA1), due to hybridization with endoglin mRNA in these cells (FIG. 4F-4H) This is consistent with the observations in the single-cell RNAseq results shown in FIG. 2.

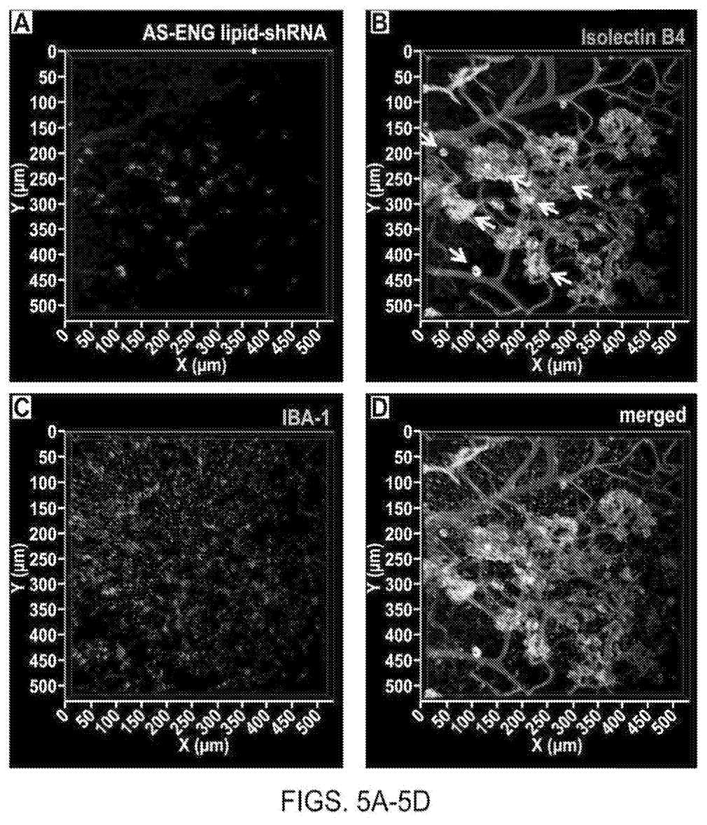

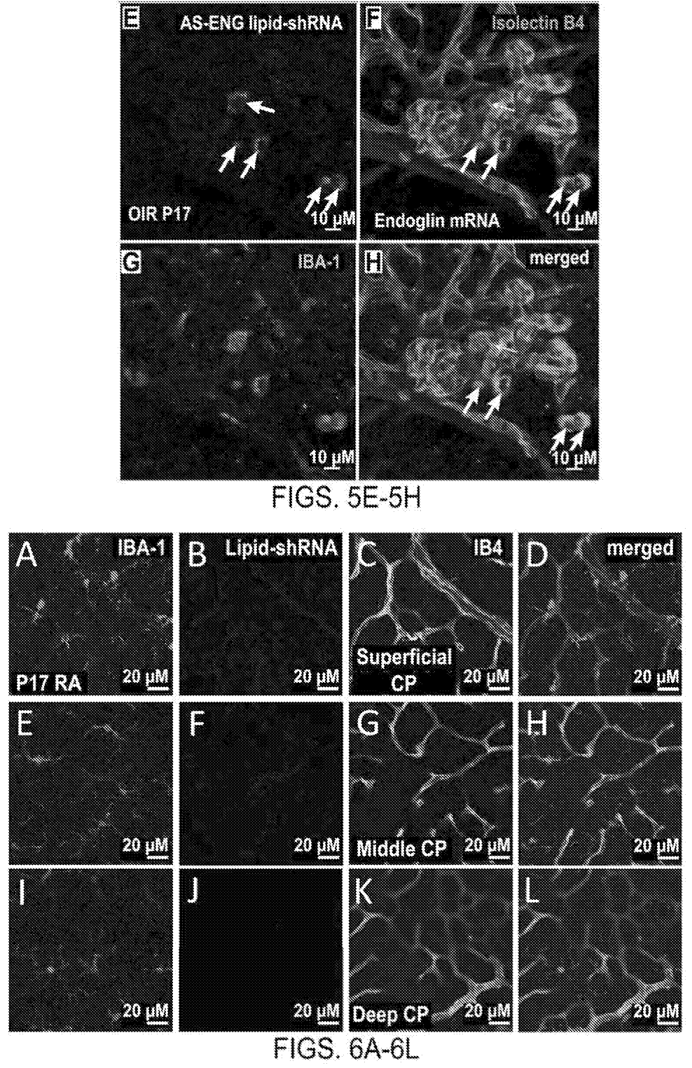

To use this method as a tool for determining neovascularization in proliferative retinopathy, the excised retina was analyzed for detailed analysis, and the results are shown in FIG. 5. Strong fluorescence (red punctate) was observed in cells that were also associated with neovascular tufts identified by structure and Isolectin B4 counterstaining. Two morphologically distinct cell populations positive for AS-shRNA-lipid-derived fluorescence that were also positive for IBA1 IBA1 was employed as a marker for retinal microglia. Interestingly, AS-shRNA-lipid-derived punctate fluorescence was observed in perinuclear regions of the non-ramified IBA1-positive cells and throughout the cytoplasm. No AS-shRNA-lipid-derived fluorescence was observed inside the nucleus of these IBA1-positive cells. Notably, no observe AS-shRNA-lipid-derived fluorescence was observed in the retinal microvascular endothelial cells in the neovascular tufts that were also positive for endoglin mRNA as shown in FIG. 3. This observation indicates that after intraperitoneal injections, shRNA-lipid conjugates were internalized into the IBA1-positive cells which then migrated to the retina. This migration can be a response to neovascularization and can be used as measure of the severity of the disease progression and also treatment response in proliferative retinal disease. To confirm the specificity of the AS-shRNA-lipid conjugate, the following control experiments were performed: the same AS-shRNA-lipid conjugate was injected to age-matched normal healthy control animals as shown in FIG. 6 where the number of MRC-1-positive cells were minimal as shown in FIG. 2. Interestingly, IBA1-positive cells were distributed around the vasculature in the superficial, middle and deep capillary plexus and were minimally positive for AS-shRNA-lipid-derived fluorescence in all three layers in healthy control retinas.

Furthermore, it was confirmed that the intracellular delivery of AS-shRNA-lipid conjugate into bone marrow derived macrophages (MMa-bm) and imaging endoglin mRNA in vitro (FIG. 9). Endoglin mRNA was induced in MMa-bm cells using phorbol myristate acetate (PMA) following a known protocol. It was observed that intracellular delivery of shRNA-lipids can be achieved in MMa-bm phagocytic macrophages and can also be induced by using poly-L-lysine (PLL). In MMa-bm cells without PMA treatment, AS-shRNA-lipid derived fluorescence was minimally observed, indicating lower expression of endoglin mRNA.

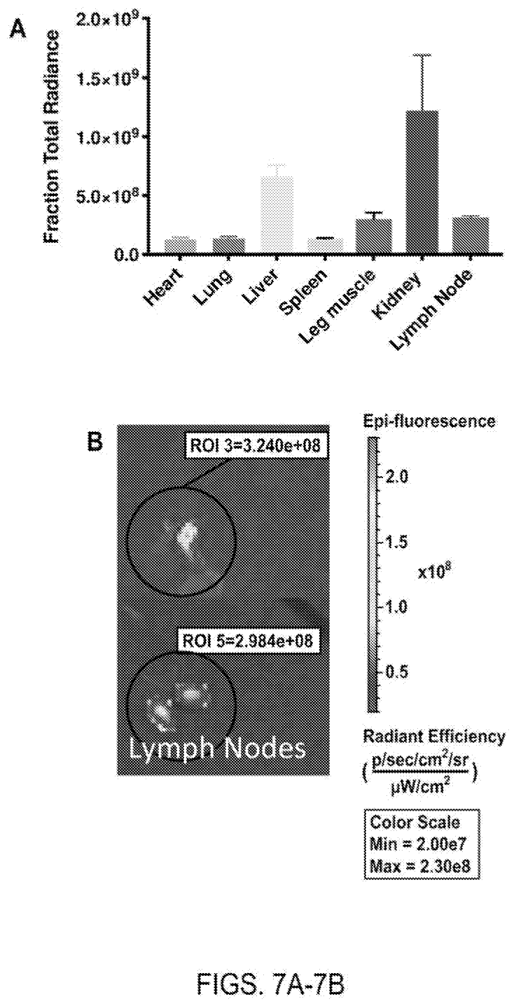

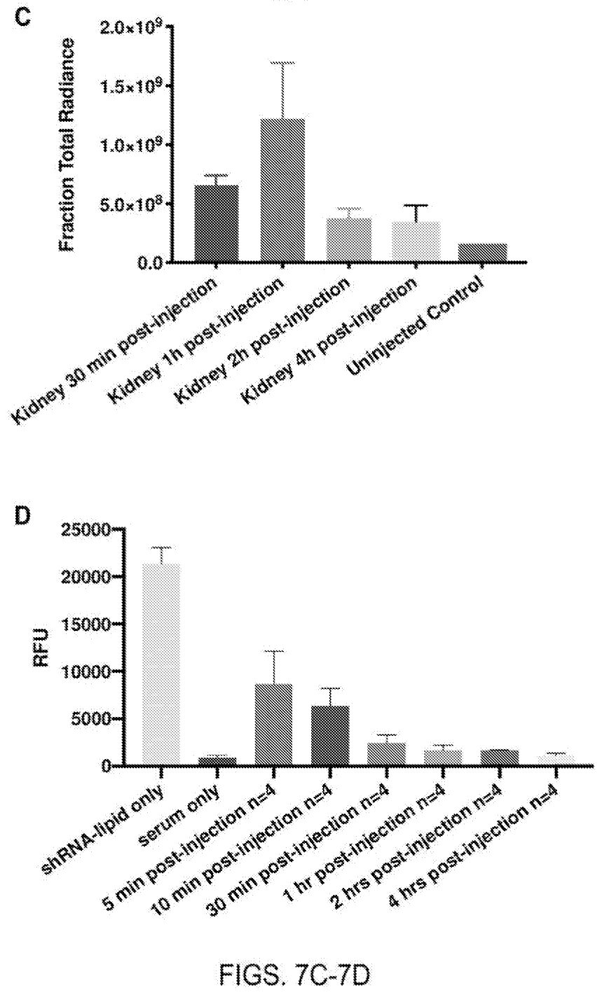

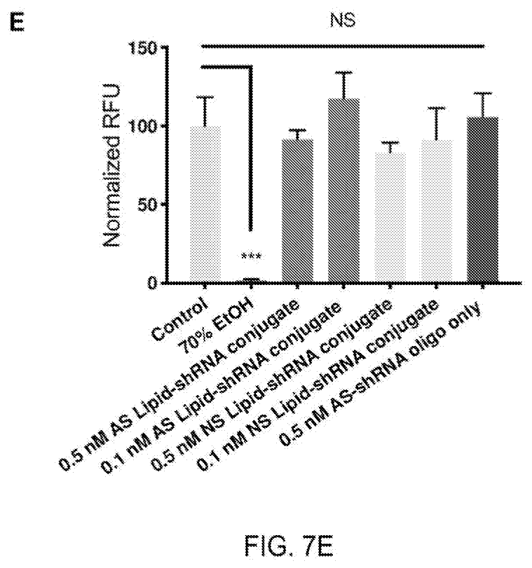

In vivo bio-distribution and toxicity of AS-ENG-shRNA-lipid conjugates. From bio-distribution analysis as shown in FIG. 7, AS-shRNA-lipid conjugate clears after 2 hours mostly through kidney and after eighteen hours post-injection, the imaging agents were localized in IBA1 positive cells in the retina. A previous study related to targeted delivery of gold-nanoparticles conjugated hairpin DNA (hAuNP) shows that hAuNP were mostly trapped in spleen and liver. However, AS-shRNA-lipids have prolonged bioavailability, more than an hour in vivo for tissue uptake; the unbound probes cleared through renal secretion mostly after 4 hours. To confirm the safety of AS-shRNA-lipid, a live-dead assay was used to monitor cell viability in vitro after treatment with AS-shRNA-lipid. A non-sense probe (NS-shRNA-lipid) was used as control to monitor any sequence specific toxicity. Mouse retinal microvascular endothelial cells (MRMECs) were incubated with AS-shRNA-lipid or NS-shRNA-lipid, and live-dead assay using Calcein AM confirmed that independent of their nucleotide sequence, shRNA-lipids were not toxic to the retinal cells.

### DISCUSSION

The mouse OIR model is widely used as an experimental model of pathologic retinal angiogenesis. This model involves exposing 7-day old mice (postnatal day 7 or “P7”) to 75% oxygen for 5 days. Hyperoxic exposure injures the retinal vasculature, giving rise to a central retinal avascular area. On P12, the mice are returned to normoxia, and the ischemic central avascular retina becomes hypoxic. Hypoxia elicits the elaboration of retinal VEGF and other vasoactive factors that trigger a robust neovascular response. Development of pre-retinal neovascularization begins at P13, rapidly advances and plateaus at P17. The neovascular tufts are observed at the border between the central avascular and peripheral vascular retina; they begin to regress slowly around P19 and are resolved by P25.

Because endoglin expression is associated with a specific population of cells in the mouse model of neovascularization as described in the Results section, it can be exploited to improve the clinical management of proliferative retinopathy as observed in PDR patients. To accomplish this, a novel imaging tool has been designed, constructed and characterized—an mRNA-targeted shRNA-lipid conjugate. The combined molecular features of this lipid conjugate offer significant advantages over other imaging probes. For example, the probe becomes fluorescent upon hybridization to target-mRNA, allowing non-invasive optical imaging in the retina. After intraperitoneal injection, this lipid conjugate is chaperoned by albumin throughout systemic circulation and is efficiently delivered to the target tissues and retained, without the need for potentially toxic transfection reagents. In the current study, AS-ENG-shRNA-lipid conjugate was used for molecular imaging of endoglin mRNA in neovascularization in mouse OIR.

Diacyl-lipid conjugated siRNA can be efficiently delivered to the lymphatic system through ‘albumin hitchhiking’ where they can be efficiently internalized into the phagocytes and increase the T-cell priming to treat cancer. In the current example, a similar strategy was applied by using stabilized short hairpin RNA (shRNA) that are conjugated to diacyl-lipid (shRNA-lipid) to image endogenous mRNA in phagocytic cells in vivo and track these cells if they were really contributing in response to retinopathy. Overall, after intraperitoneal injection, the lipid moiety of the AS-shRNA-lipid conjugates protects the shRNA from degradation and blocks off-target extracellular interactions. This allows for efficient delivery of the conjugates to the lymphatic system through ‘albumin hitchhiking’ where they can be efficiently internalized into the IBA1 positive phagocytes tagging endoglin mRNA and then migrated to the neovascular tufts in the OIR retina. The activated microglial cells were imaged in the retina 18 hours post intraperitoneal (IP) injection. After IP injection, the shRNA-lipid conjugates are trapped in these activated cells for a very long time and can even degrade and can create non-specific fluorescence signals in vivo. The present method can detect specific activated cells by targeting mRNA biomarker to predict the ‘onset’ of neovascularization, a common complication observed in proliferative retinopathies including proliferative diabetic retinopathy (PDR) and retinopathy of prematurity (ROP). No shRNA-lipid derived fluorescence was observed in healthy age-matched control retinas where the number of activated phagocytic cells are minimal as shown in FIG. 2. To test the specificity of the shRNA-lipid in macrophages, endoglin mRNA was induced in bone marrow derived macrophages and the AS-shRNA-lipid was used to confirm its specificity and sensitivity in vitro. The data show that shRNA-lipid can be internalized into the phagocytic cells and can fluoresce when the target mRNA was induced in these cells.

### Methods

All chemicals were purchased form Sigma-Aldrich (St. Louis, Mo.) and used as received unless otherwise noted. The mouse primary retinal microvascular endothelial cells (MRMEC) were obtained from Cell Biologics Inc (IL, USA). Mouse macrophages from C57BL/6 bone marrow (MMa-bm) were obtained from ScienceCell (CA, USA). Custom designed 2′-O-methyl-protected short hairpin RNA (shRNA) and custom oligonucleotides were custom synthesized from Integrated DNA Technologies Inc. (IA, USA).

Animals: Multi-timed pregnant C57BL/6J female mice were purchased from Charles River Laboratories. All animal procedures used in this study were approved by the Vanderbilt University Institutional Animal Care and Use Committee and were performed in accordance with the Association for Research in Vision and Ophthalmology Statement for the Use of Animals in Ophthalmic and Vision Research.

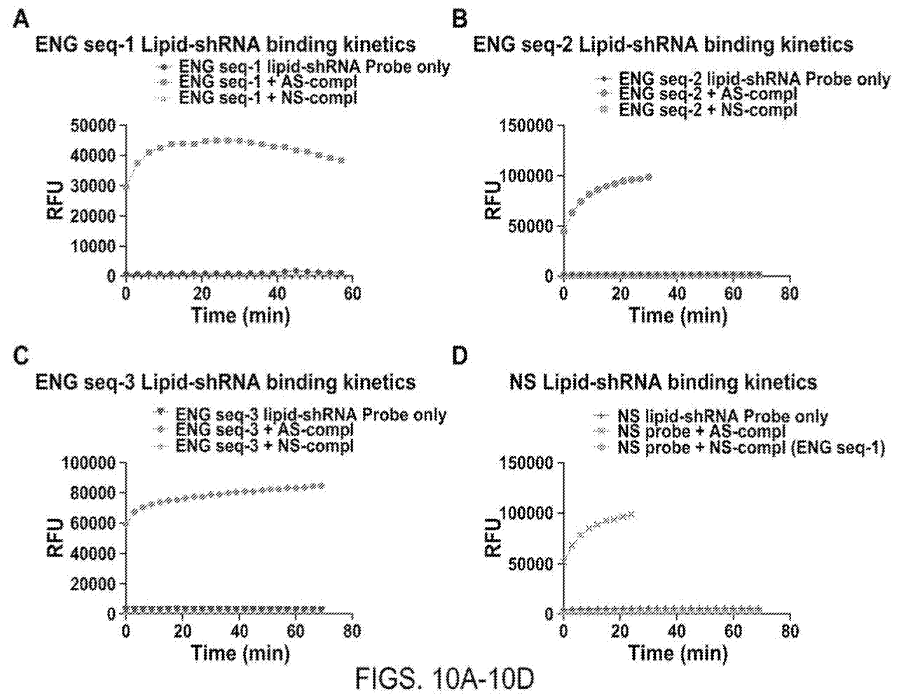

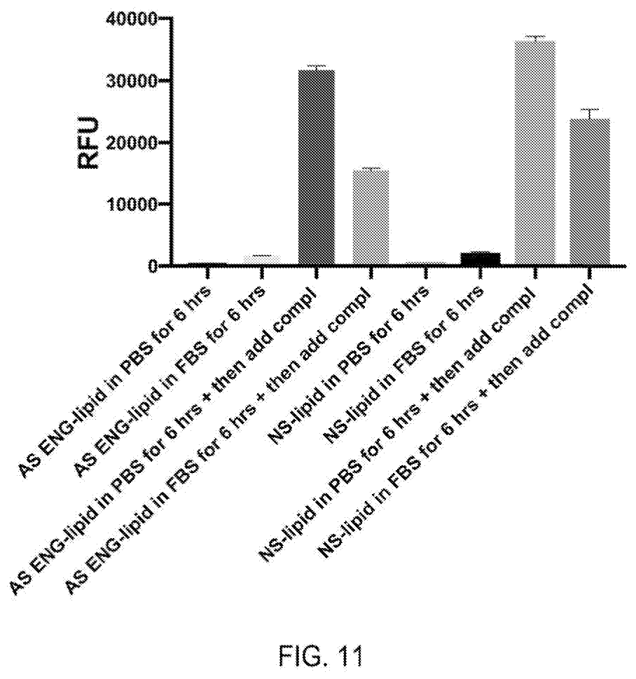

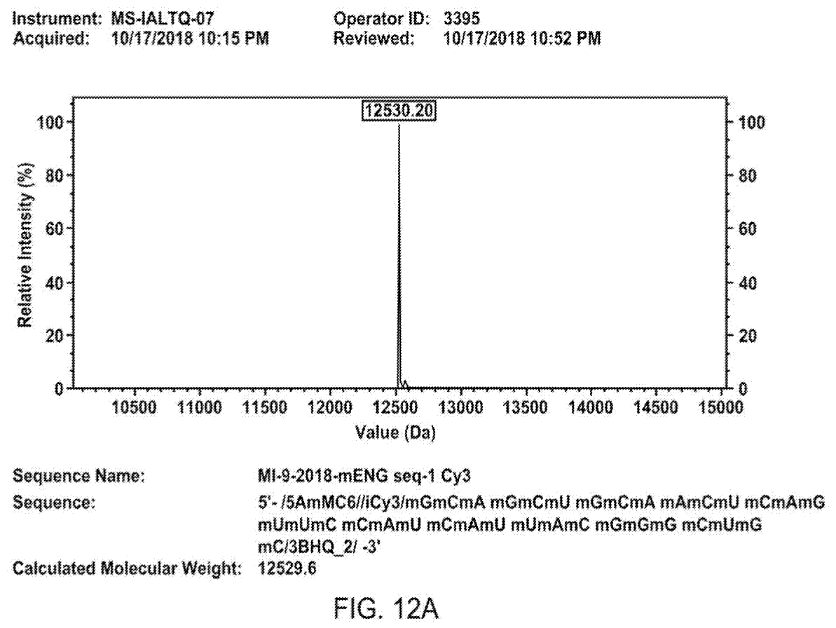

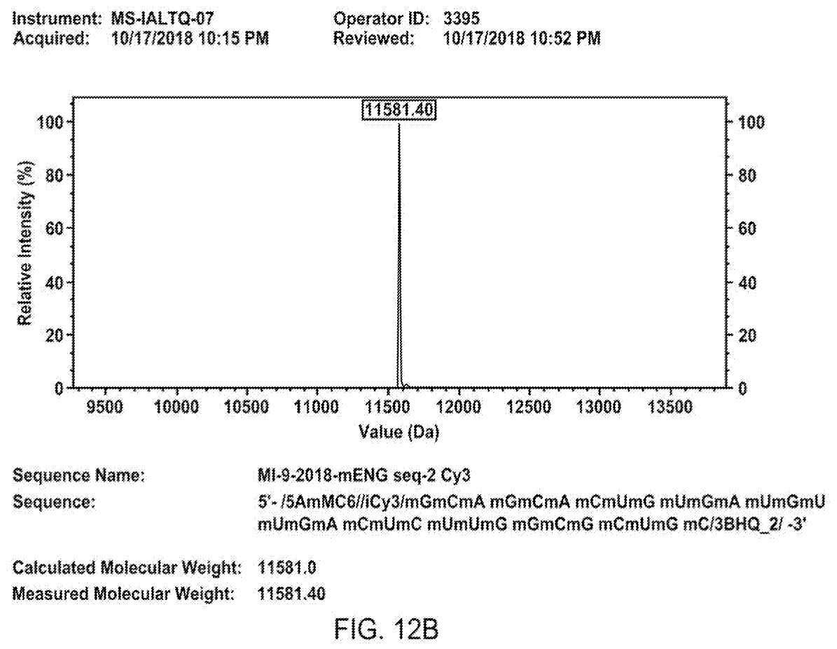

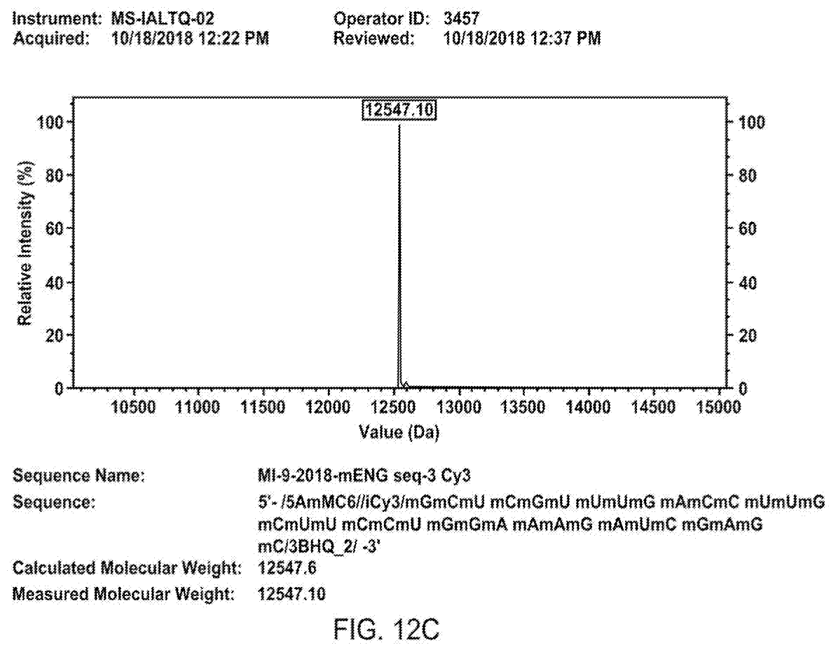

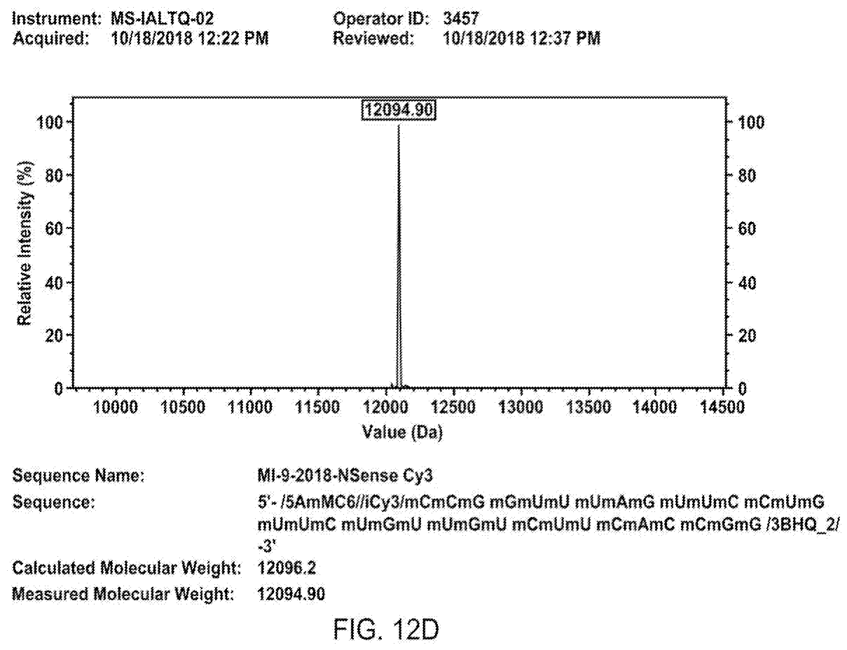

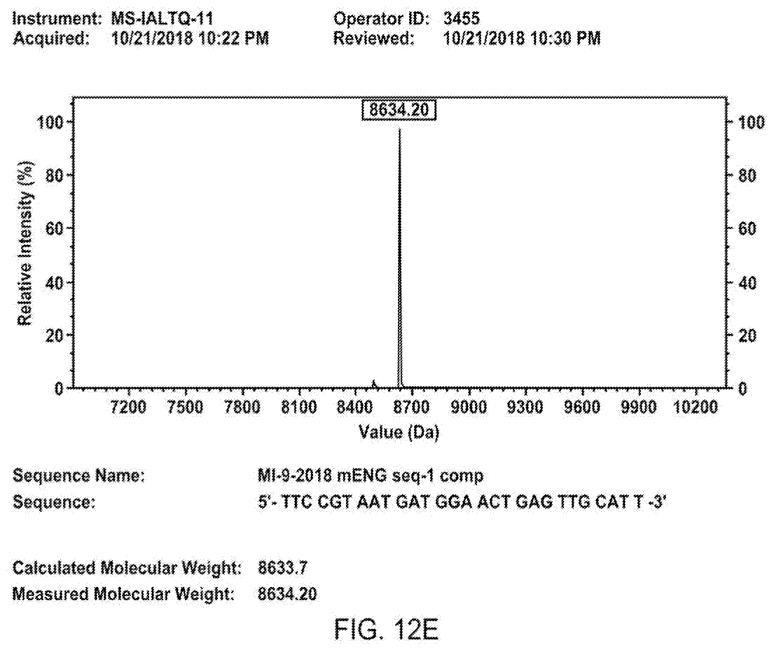

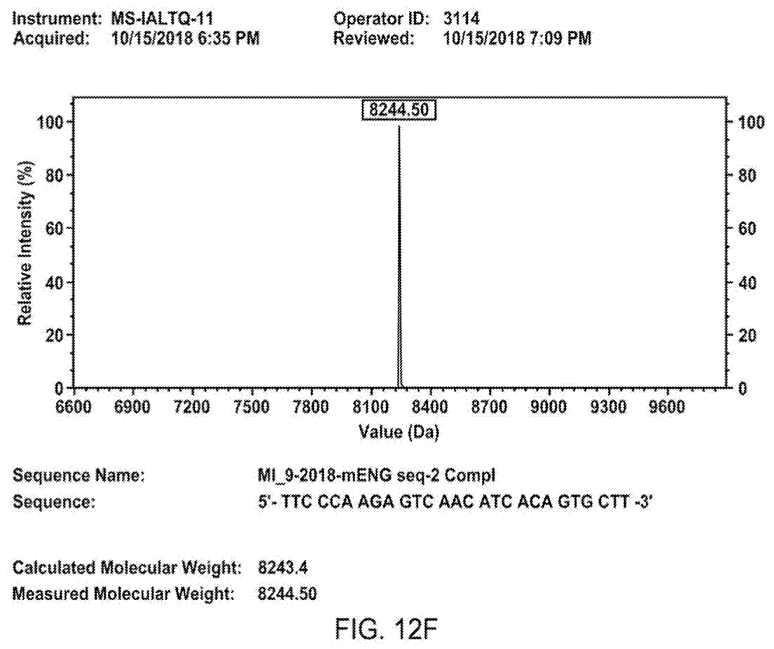

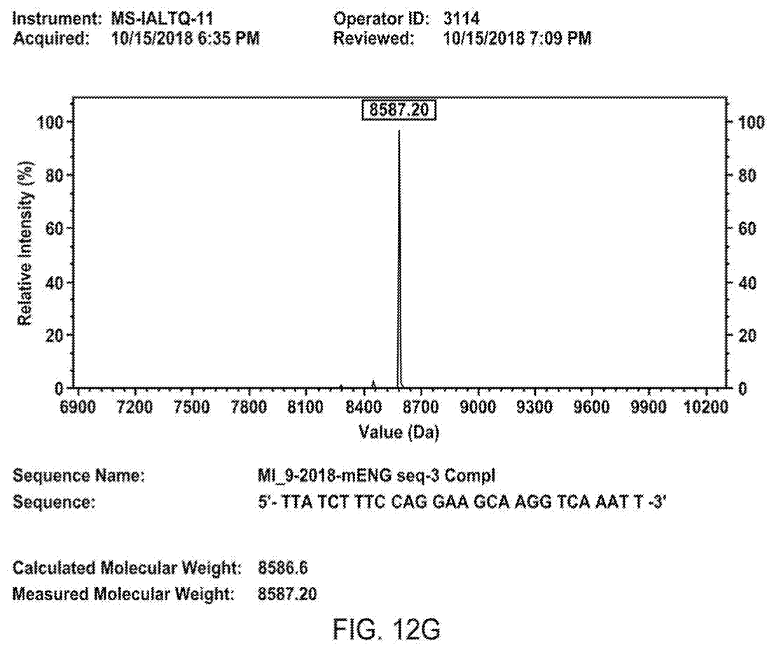

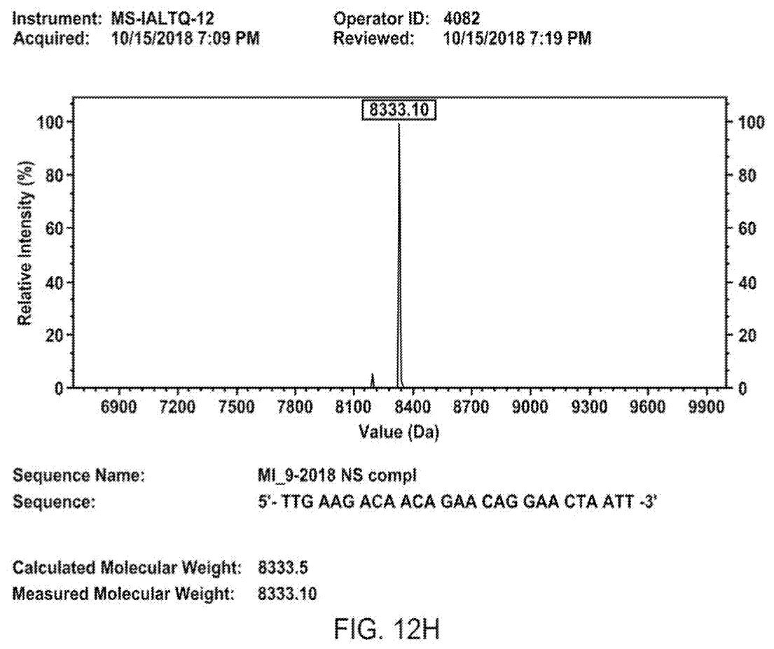

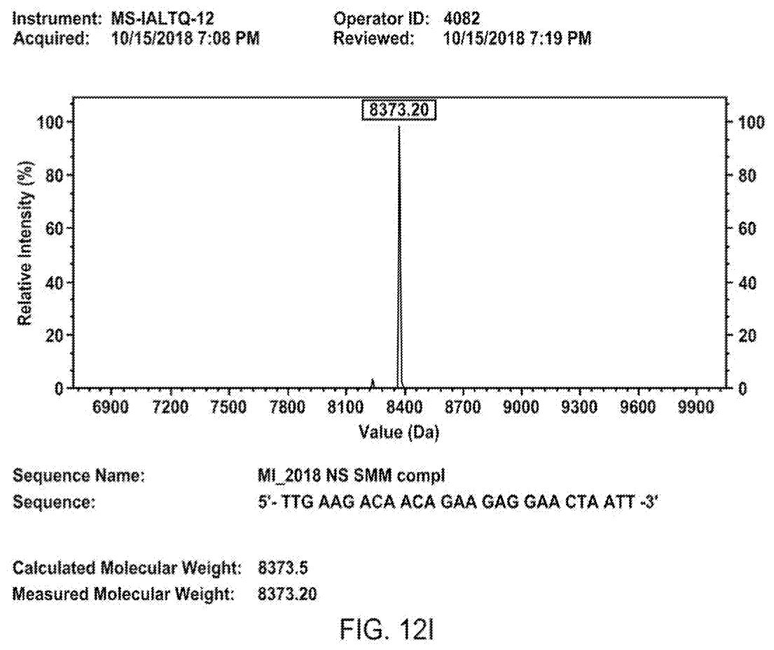

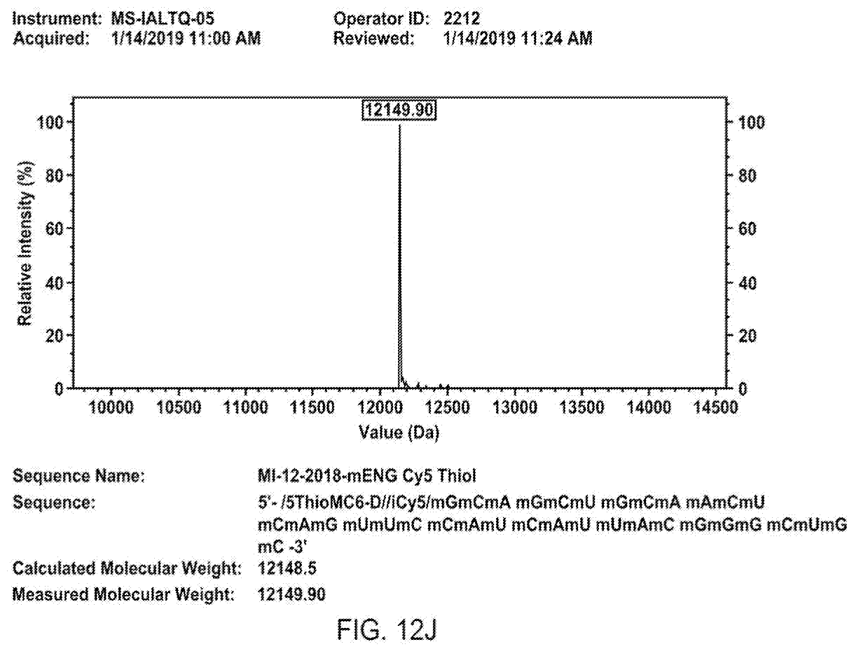

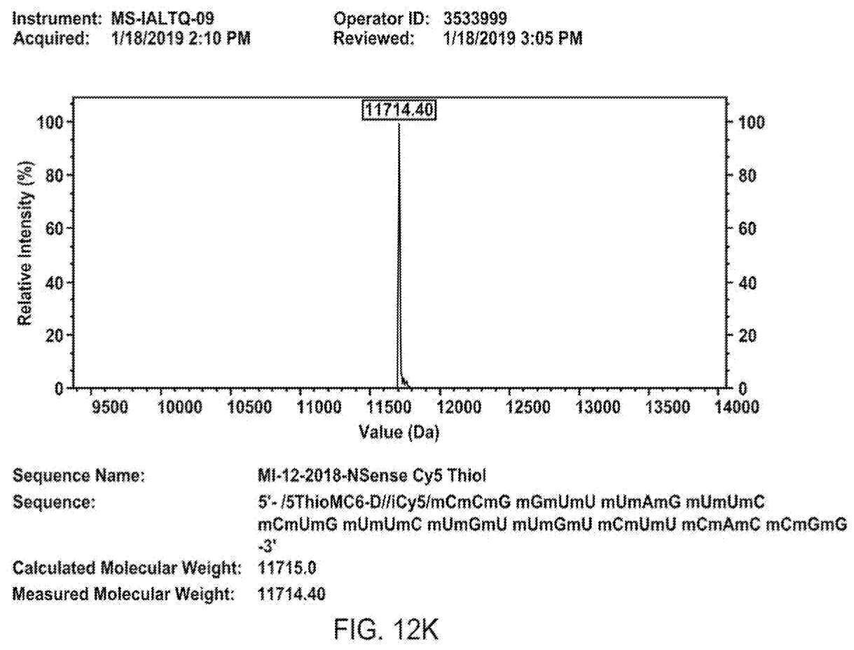

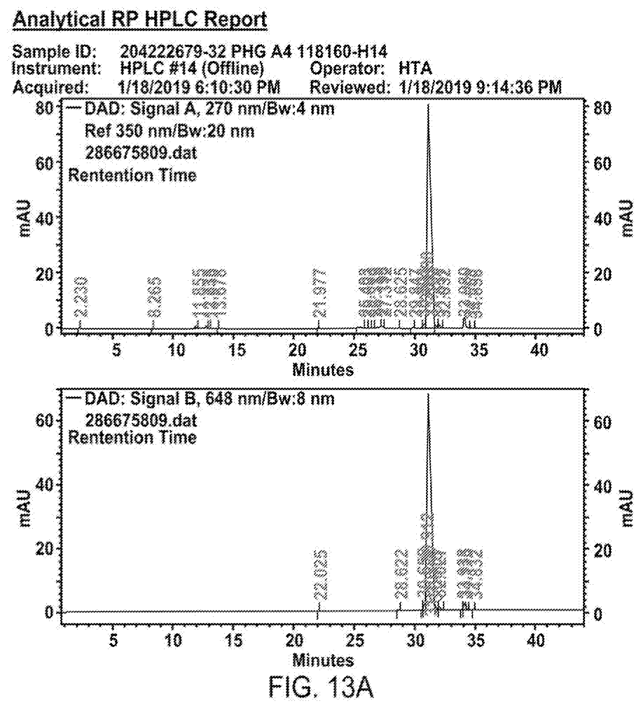

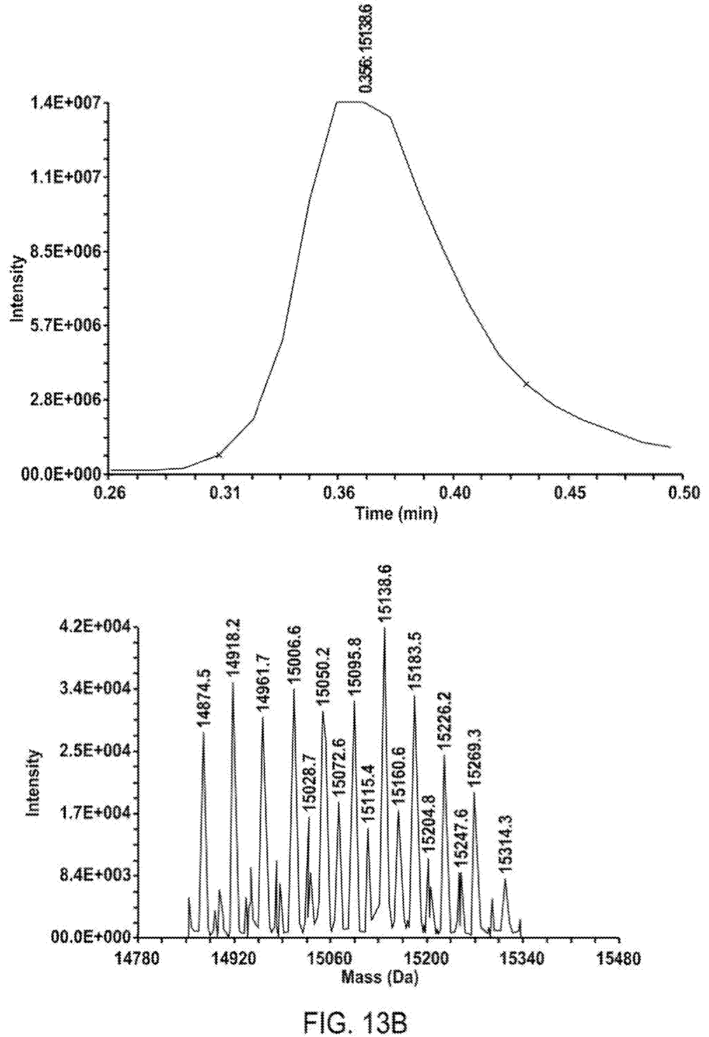

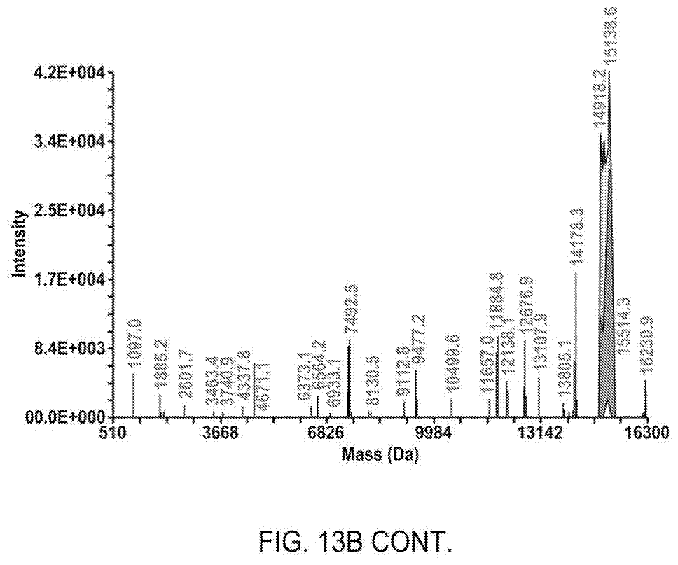

Design and synthesis of AS-ENG-shRNA-lipid conjugates: The 2′-O-methyl-protected short hairpin RNA (shRNA) oligonucleotides that incorporate mouse ENG mRNA specific sequences were synthesized and purified using HPLC system. The anti-sense ENG sequences (SEQ ID mENG seq-1, SEQ ID mENG seq-2, SEQ ID mENG seq-3 as shown below) were extensively BLAST searched to determine no significant overlap with any other mouse mRNA transcript. The anti-sense ENG sequences are located within the loop of the hairpin structure as shown in FIG. 1. A self-complementary sequence was incorporated to form the stem of the shRNA hairpin. This sequence is largely responsible for the formation and the stability of the hairpin secondary structure. Each shRNA was computationally designed via energy minimization to achieve the formation of the hairpin structure. Each of the shRNA-oligonucleotides was coupled to a Cy3 dye (fluorophore) and C6 amino group to facilitate conjugation to the diacyl-lipid. The 3′ end was coupled to a BHQ2. Finally, the shRNA was conjugated to the diacyl-lipid according to a previously described method with slight modification. Briefly, amine-functionalized hairpin-shaped RNA was reacted with 10-fold molar excess of dibenzocyclooctyne-PEG₄-N-hydroxy-succinimidyl ester (DBCO-PEG₄-NHS) predissolved at 25 mM in DMSO. The reaction was carried out for overnight at room temperature at a 1 mM oligonucleotide concentration in 30% DMSO and 70% PBS with 8 mM diisopropylethylamine (DIPEA). The product was then diluted threefold in water and the excess reagents were removed by centrifugal filtration using a filter with a 3 kDa molecular weight cut-off (Amicon Ultracel 10K from Millipore, Billerica, Mass.) and washed twice using PBS (Life Technologies Corporation; Grand Island, N.Y.), and then reacted with fivefold molar excess of DSPE-PEG₂₀₀₀-azide for 24 h at a 0.1 mM oligonucleotide concentration in 50% methanol, 50% water. The reaction was then diluted in water then purified using 10 kDa (Amicon Ultracel 10K from Millipore, Billerica, Mass.) molecular weight cut-off filter and washed three times using PBS. The pure conjugate was collected and diluted in PBS for characterization and in vivo applications. Molecular weight was confirmed using MALDI-TOF mass spectrometry (Voyager-DE STR Workstation) using 50 mg/mL 3-hydroxypicolinic acid in 50% water, 50% acetonitrile with 5 mg/mL ammonium citrate as a matrix or ESI-TOF MS analysis. The freshly conjugated lipid-oligonucleotide spontaneously forms nanoparticles and were stored at 4° C. until used. After complete synthesis, specificity and sensitivity of the purified AS-shRNA-lipid conjugates were examined using complementary sequence and compared with nonsense complementary sequence as shown in FIG. 10. Among several candidate sequences, mENG seq-2 was highly responsive in presence of complementary sequence and selected to use for molecular imaging of target mRNA in vivo. AS-shRNA-lipid as well a non-sense shRNA-lipid conjugates have similar stability profiles in serum containing medium (FBS) for at least 6 hours at 37° C. (FIG. 11), indicating their stability in vivo. In addition, the probes can hybridize to their corresponding complementary sequence as observed from increased fluorescence, indicating their retained hairpin structures after incubation in FBS for 6 hours.

The shRNA sequence of the AS-ENG-shRNA is shown as:

mG means 2′-OMe protected G, mC means 2′-OMe
protected C, mA means 2′-OMe protected A,
mU means 2′-OMe protected U:
SEQ ID mENG seq-1 Cy3:
(SEQ ID NO: 5)
5′-mGmCmAmGmCmUmGmCmAmAmCmUmCmAmGmUmUmCmCmAmUmCmAm
UmUmAmCmGmGmGmCmUmGmC-3′.
SEQ ID mENG seq-2 Cy3:
(SEQ ID NO: 1)
5′-mGmCmAmGmCmAmCmUmGmUmGmAmUmGmUmUmGmAmCmUmCmUmUm
GmGmCmGmCmUmGmC-3′
SEQ ID mENG seq-3 Cy3:
(SEQ ID NO: 6)
5′-mGmCmUmCmGmUmUmUmGmAmCmCmUmUmGmCmUmUmCmCmUmGmGm
AmAmAmGmAmUmCmGmAmGmC-3′
SEQ ID for NS sequence Cy3:
(SEQ ID NO: 10)
5′-mCmCmGmGmUmUmUmAmGmUmUmCmCmUmGmUmUmCmUmGmUmUmGm
UmCmUmUmCmAmCmCmGruG-3′
Sequence positions in target mRNA:
For ENG seq-1 Mus musculus endoglin (Eng),
transcript variant 1, mRNA NM_007932.2:
(SEQ ID NO: 7)
756 GCCAAGAGTCAACATCACAGTGCT 779
For ENG seq-2 Mus musculus endoglin (Eng),
transcript variant 1, mRNA NM_007932.2:
(SEQ ID NO: 8)
1073 CCGTAATGATGGAACTGAGTTGCA 1096
For ENG seq-3 Mus musculus endoglin (Eng),
transcript variant 1, mRNA NM_007932.2:
(SEQ ID NO: 9)
1204 ATCTTTCCAGGAAGCAAGGTCAAA 1227

Dynamic light scattering (DLS): DLS measurements were performed on a Malvern Zetasizer Nano ZS (Malvern Instruments, Inc.; Westborough, Mass.). Particle measurements were performed at a concentration of 10 μM shRNA-lipid in PBS (Life Technologies Corp.; Carlsbad, Calif.). These measurements were performed in triplicate.

Transmission electron microscopy (TEM): shRNA-lipid probes were mounted on 300-mesh copper grids and stained with 2% uranyl acetate. Samples were subsequently imaged on the Philips/FBI Tecnai T12 electron microscope (Hillsboro, Oreg.) at various magnifications.

In vivo and ex vivo imaging of mRNA in mouse OIR: To generate the OIR mouse model, dams with their pups were treated with 75% oxygen for 5 days from postnatal day 7 (P7) to P12. On P12, pups were removed from the hyperoxic chamber and stayed with the nursing mother in normal air condition for additional 5 days. At P17 AS-shRNA-lipid conjugates in sterile saline were injected intraperitoneally at a dose of 0.5 mg/kg. After 18 hours, AS-shRNA-lipid dependent fluorescence imaging was performed in vivo. Briefly, mice were anesthetized with ketamine/xylazine, eyes were dilated with 1% tropicamide, and fluorescent images were acquired using a confocal scanning-laser microscopy-imaging system (LSM 710 META Inverted, Jena, Germany). Then, ex vivo fluorescence imaging was performed to localize AS-shRNA-lipid derived fluorescence in ocular tissues in OIR retina. After imaging, animals were sacrificed, enucleated and the globes were fixed in 10% neutral buffered formalin (NBF). Retinas were dissected and blocked/permeabilized in 10% donkey serum with 1% Triton X-100 and 0.05% Tween 20 in TBS for 2 hours and were then counter-stained for IBA1 and IB4 conjugated to Alexafluor-dyes (Life Technologies; Grand Island, N.Y.). The tissues were then mounted with Prolong Gold mounting medium with DAPI (Life Technologies; Grand Island, N.Y.). Images were taken using an epifluorescence Nikon Eclipse Ti-E inverted microscope (Melville, N.Y.).

In vivo biodistribution of plasma half-life measurement of shRNA-lipid conjugates: For biodistribution assays, 4 to 6 weeks old adult C57BL/6J mice were used. Cy3-shRNA-lipid (without the quencher) conjugates in sterile saline were injected intraperitoneally at a dose of 0.5 mg/kg. Blood samples were collected for plasma half-life measurements from deeply anesthetized animals at 0 min, 5 min, 10 min, 0.5 hr, 1 hrs, 2 hrs, and 4 hrs (n=4 for each time point, duplicate experiments). Blood samples were taken by cardiac puncture into a heparinized syringe into a 1.5 ml heparinized tube on ice, followed by removal of the heart, lung, liver, kidney, spleen, leg muscle and lymph nodes. The fluorescence intensity in the organs were quantified using Xenogen IVIS 200 fluorescence imaging system (PerkinElmer, USA) at excitation wavelength of 550±5 nm and emission wavelength of 570±5 nm (n=4 animals) The blood samples were centrifuged at 6000 rpm for 5 min. Plasma samples were transferred to a clean tube and fluorescence intensities were measured using Synergy MX microplate reader (BioTek, USA) at excitation wavelength of 550±5 nm and emission wavelength of 570±5 nm (n=4).

Isolation of bone marrow-derive cells from mouse OIR and RA pups: Peripheral blood samples were collected from deeply anesthetized OIR and age-matched RA control pups. Bone marrow-derive mononuclear cells were isolated from fresh blood within 2 hours of collection, using Ficoll-Paque density gradient centrifugation as described.³⁷

Single cell RNAseq: Each sample (targeting 5,000 cells/sample) was processed for single cell 5′ RNA sequencing utilizing the 10× Chromium system. Libraries were prepared using P/N 1000006, 1000080, and 1000020 following the manufacturer's protocol. The libraries were sequenced using the NovaSeq 6000 with 150 bp paired end reads. RTA (version 2.4.11; Illumina) was used for base calling and analysis was completed using 10× Genomics Cell Ranger software v2.1.1. or Loupe Cell Browser.

Cell Culture: MRMECs were cultured in T-75 cell culture flasks (Thermo Fisher Scientific; Wilmington, Mass.) coated with attachment factor (Cell Systems; Danvers, Mass.) and in growth medium consisting of endothelial basal medium (EBM; Lonza; Walkersville, Md.) supplemented with 2% FBS (Lonza) and endothelial cell growth supplements (EGM SingleQuots; Lonza). MMa-bm cells were cultured according to the supplier's instruction in complete MaM growth medium. All cultures were incubated at 37° C., 5% CO2 and 95% relative humidity (20.9% oxygen). The cells were cultured in 95-well plates and treated with shRNA-lipid in complete growth medium. Passages 4 to 6 were used to assess the toxicity of the imaging probes.

In vitro hybridization of AS-ENG-shRNA-lipid: To determine target specificity, anti-sense ENG-shRNA-lipid conjugate (1 nM in sterile PBS) was titrated with ENG-recognition complementary sequence (AS-compl) or nonsense oligo (NS-compl) at a concentration (0.1 μM). Fluorescence intensities were measured using a microplate reader (Biotek, Winooski, Vt.) and plotted as a function of time. Signal to noise was determined by the fluorescence ratio of the AS-compl vs the NS-compl. Experiments were performed at least three times with n=3 for each experimental group.

Fluorescence in situ hybridization (FISH): Formalin-fixed paraffin-embedded (FFPE) OIR mouse eyes were sectioned in 6-μm thick slices and were deparaffinized in xylene, followed by dehydration in an ethanol series. Tissue sections were then incubated in citrate buffer (10 nmol/L, pH 6) maintained at a boiling temperature (100° C. to 103° C.) using a hot plate for 15 minutes, rinsed in deionized water, and immediately treated with 10 μg/mL protease plus reagent (Advanced Cell Diagnostics, Hayward, Calif.) at 40° C. for 30 minutes in a HybEZ hybridization oven (Advanced Cell Diagnostics, Hayward, Calif.). Hybridization with target probes, preamplifier, amplifier, and fluorescence detection using TSA Plus fluorescence detection kit (PerkinElmer, Hopkinton, Mass.) were performed in multistep procedures according manufactures instruction (Advanced Cell Diagnostics, Hayward, Calif.). Assays were performed in parallel with positive and negative controls, to ensure interpretable results. The endogenous housekeeping marker PPIB (Advanced Cell Diagnostics, Hayward, Calif.) was used as positive control to assess both tissue RNA integrity and assay procedure. The bacterial gene DapB (Advanced Cell Diagnostics, Hayward, Calif.) was used as negative control to assess background signals.

Cell viability assay: An EZViable Calcein AM Fluorometric Cell Viability Assay Kit (BioVision, Milpitas, Calif., USA) was used to quantify the number of viable cells. MRMECs were cultured on sterile black 96 well plates under growth conditions. At 75% confluence, MRMECs were treated with 0 to 0.5 nM AS-ENG-shRNA-lipid in complete medium, 0 to 0.5 nM NS-shRNA-lipid in complete medium or 70% ethanol as a positive control for 8 hrs. After treatment, the cells were washed with cold PBS (Life Technologies Corporations). MRMECs were then exposed to a buffered (1:500) calcein AM solution and incubated at 37° C. for 30 minutes. Fluorometric readings were performed using a microplate reader (Biotek; Winooski, Vt.). Fluorescence intensity was plotted on the Y-axis and represented as % live cells. Experiments were performed at least three times with n=3 for each experimental group.

Statistics. Data were expressed as mean±% SDM and statistical differences among groups were determined by one-way analysis of variance (ANOVA) using Prism 6 (Graph-Pad, San Diego, Calif.) followed by Bonferroni post hoc test to determine significant differences between specific groups. A ‘p’ value <0.05 was considered statistically significant.

### Example 2. Visualizing HIF-1α mRNA in a Sub-Population of Bone Marrow Derived Cells to Predict Retinal Neovascularization

Neovascularization (NV) is a common complication in all proliferative retinopathies, including diabetic retinopathy (DR), retinopathy of prematurity (ROP) and retinal vein occlusion (RVO). Though, the pathogenesis of neovascularization is largely unknown, ischemia-induced retinal hypoxia and the release of hypoxia-dependent vascular endothelial growth factor (VEGF), in addition to other vasoactive and/or proinflammatory factors are the central importance. Circulating endothelial progenitor cells and macrophages migrate into the retina in response to neovascularization. However, the exact role of these migratory cells and macrophages in neovascularization is largely unknown. In response to neovascularization such as that occurring in proliferative retinopathies, microglia become activated, releasing pro-angiogenic and pro-inflammatory mediators that may contribute to neovascularization. Reports have shown that adult myeloid progenitor cells migrate to the avascular retina to facilitate revascularization in a mouse model of oxygen-induced retinopathy (OIR). Precisely, myeloid-specific hypoxia-inducible factor 1 alpha (HIF-1a) expression is required for this effect. The present study shows that visualizing mRNA selectively in these migratory ‘activated’ bone marrow-derived cells can be a powerful method to predict the onset, progression and resolution of retinal neovascularization. However, visualizing mRNA specifically in these small numbers of activated cells in the retina remains challenging.

In situ hybridization (ISH) is a powerful method to visualize intracellular mRNA localization in excised tissues. However, in situ hybridization methods require the use of fixed tissues or endogenously labelled target mRNA for imaging and tracking. In a recent report, it has been shown that gold-mediated targeted delivery of oligonucleotides facilitates the real-time imaging of mRNA in living cells. In this example, anti-sense probes have been designed and synthesized to conjugate to diacyl-lipids (AS-shRNA-lipid) for targeted imaging of HIF-la mRNA that are associated with bone marrow derived cells in retinal neovascularization without using any added toxic transfection reagents.

### Results

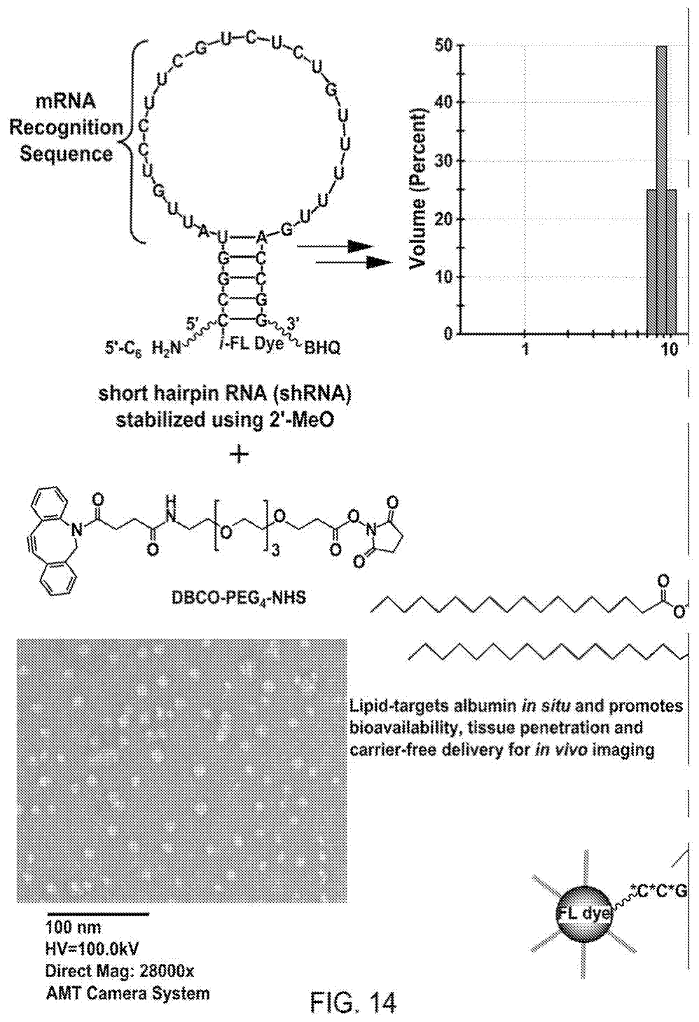

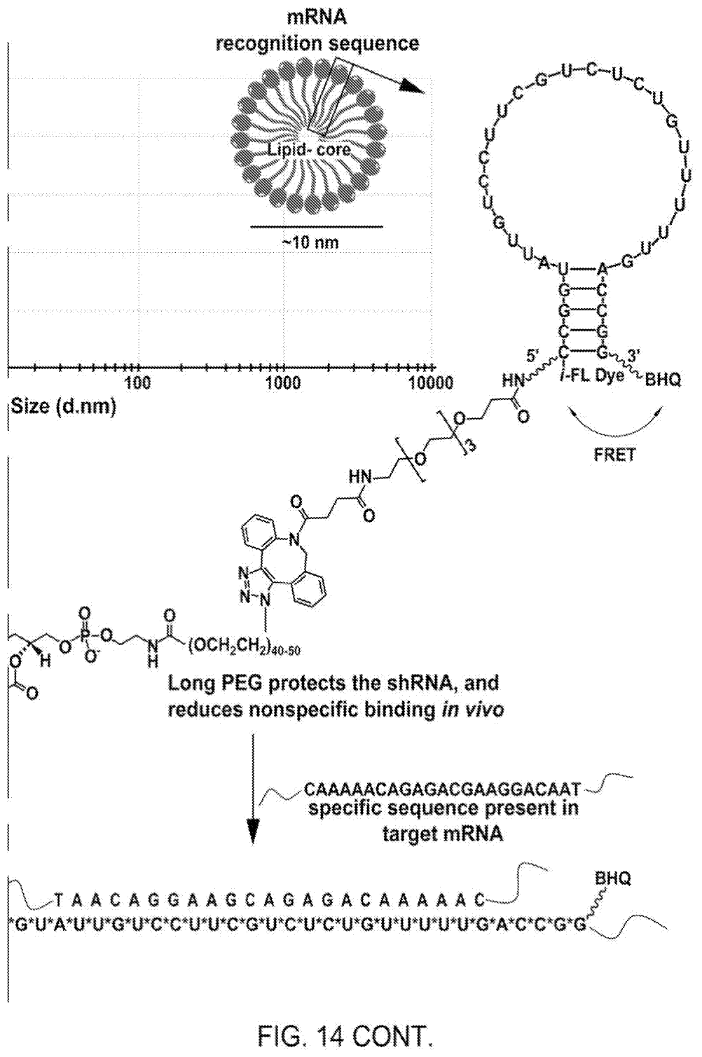

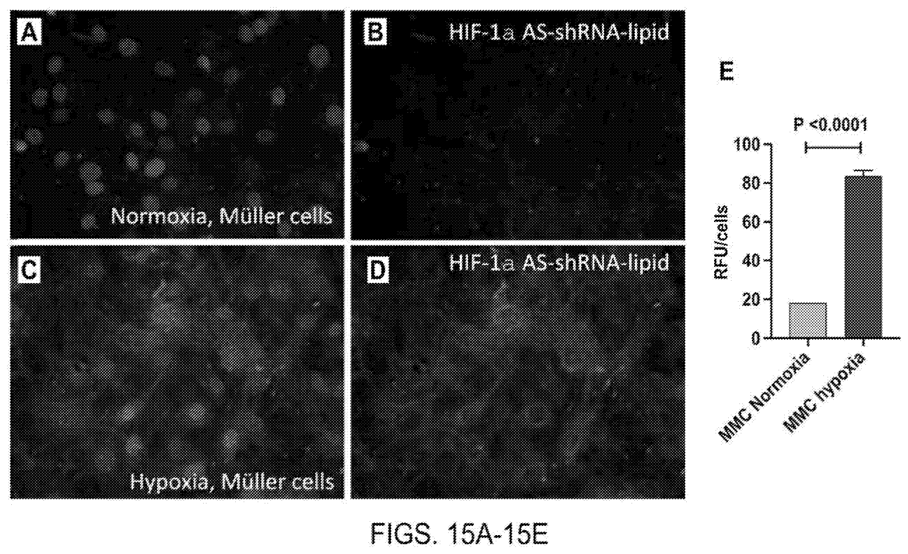

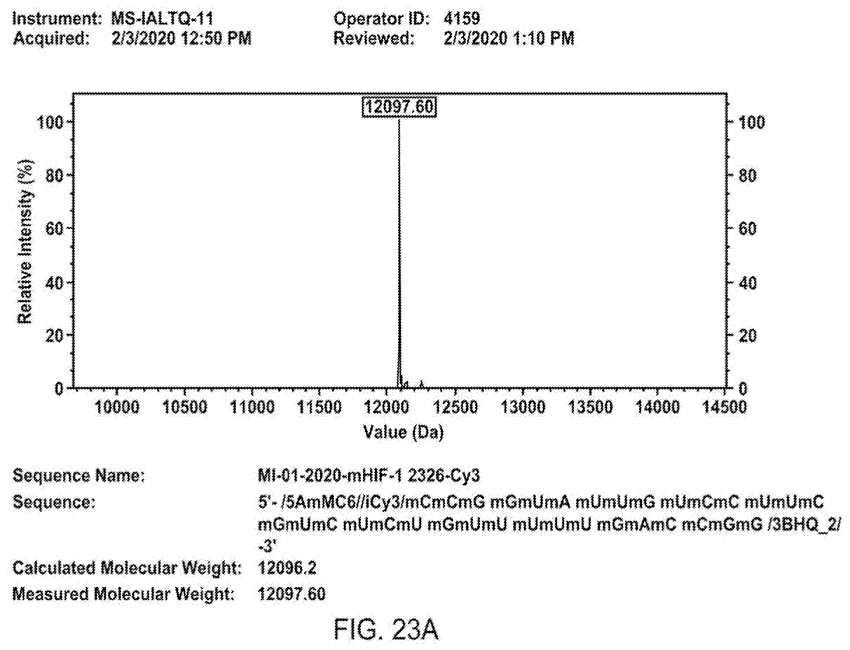

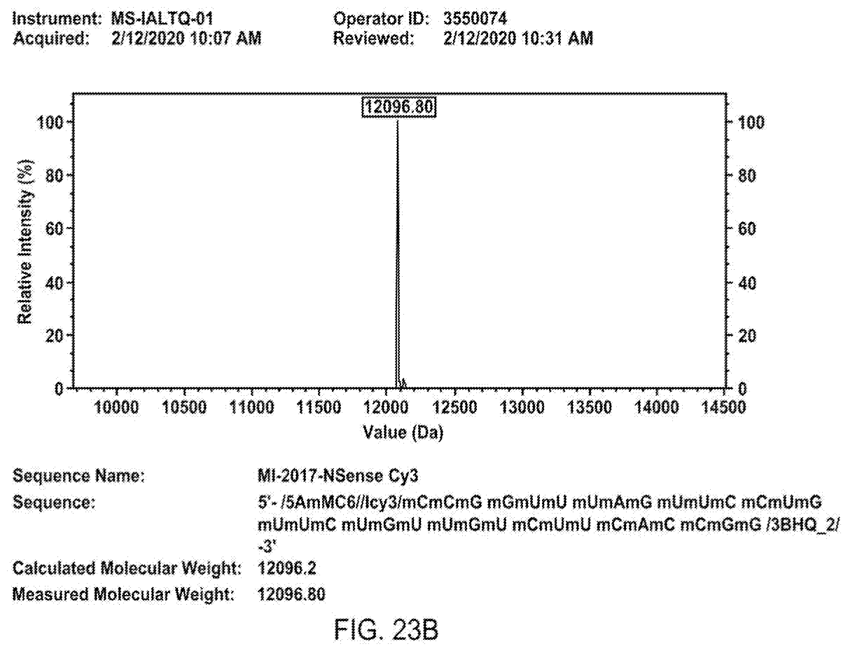

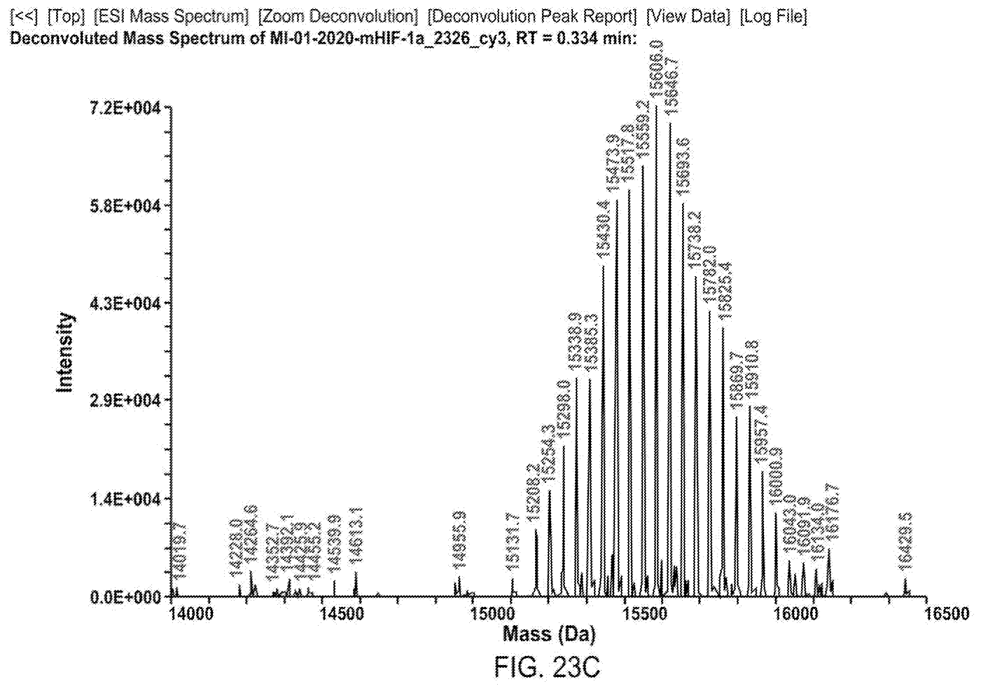

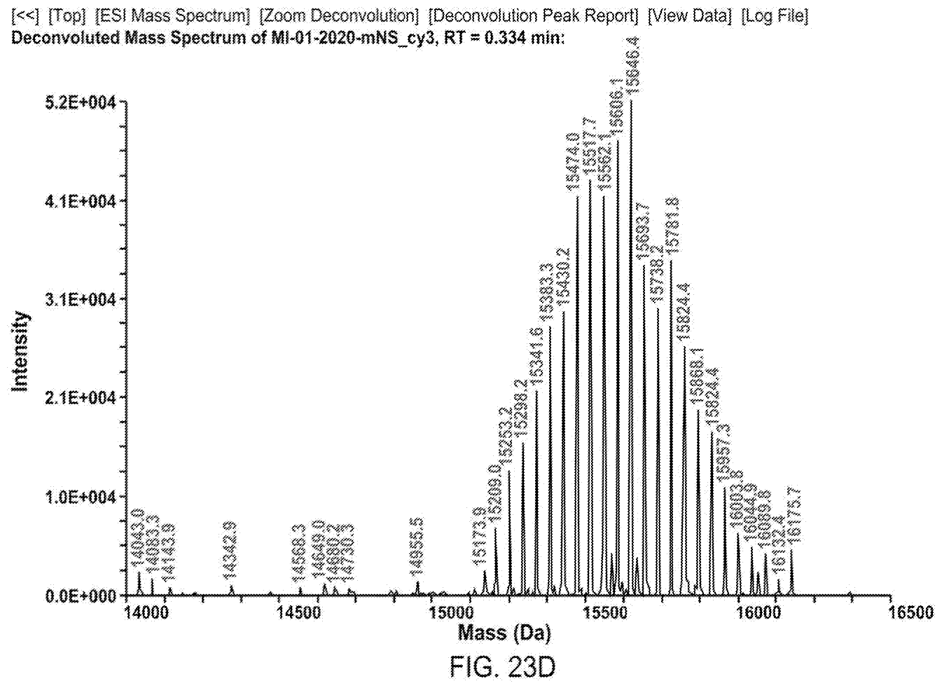

Design and synthesis of shRNA-lipid conjugates. Short-hairpin RNA (shRNA) oligonucleotides were designed computationally and synthesized using an automated solid phase synthesizer. For increased stability of the shRNA, 2′-O-methylribonucleotides (2′-OMe) were used for the oligonucleotide synthesis. A fluorescence dye at the 5′ end and a quencher (black-hole quencher or BHQ) at the 3′ end of the oligonucleotide were incorporated. Lipid conjugates were synthesized in two additional steps as described in the method section. After purification, the pure shRNA-lipid conjugates are likely to spontaneously form lipid-micelle structures in isotonic solution as shown in FIG. 14. From the dynamic light scattering (DLS) measurements of the shRNA-lipid conjugates and transmission electron microscopy (TEM) imaging, it was observed that shRNA-lipids from spherical nanoparticles of around 10 nm. The polydispersity index (PDI) value of >0.2 indicates a broad size range within the population. This multi-species nanoformulation can be due to a series of PEG lengths as observed in ESI-TOF MS data of the shRNA-lipid conjugates as shown in FIG. 23 contributing to high PDI. However, the mRNA recognition moiety remains on the surface of these nanoparticles and the diacyl-lipid-core remains inside, allowing direct hybridization with specific sequence in target mRNA as shown in FIG. 15.

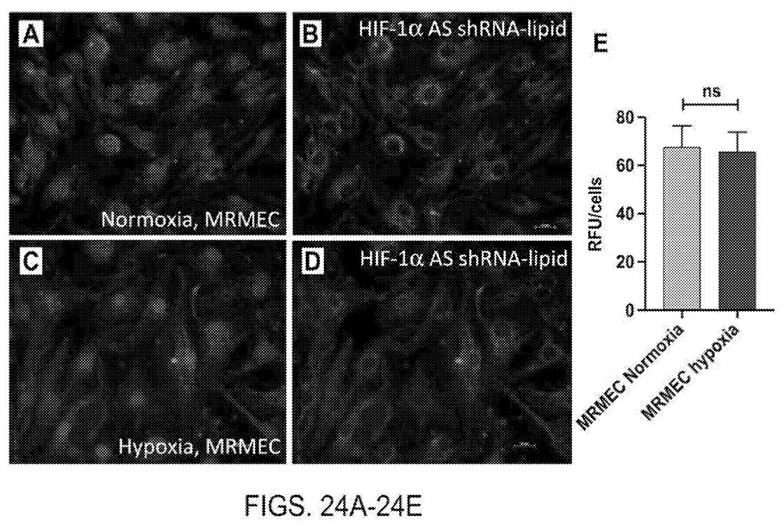

In vitro imaging of HIF-1α mRNA using shRNA-lipid. AS-shRNA-lipid was used for imaging HIF-1α mRNA expression in murine Müller cells (MMC) and primary retinal microvascular endothelial cells (MRMEC). Cells were treated under hypoxia and normoxia conditions in presence of AS-shRNA-lipid to monitor HIF-1α mRNA expression. AS-shRNA-lipid derived fluorescence was minimally detectable in normoxic MMCs and fluorescence was significantly increased in hypoxic MMCs (FIG. 15), indicating that HIF-1α mRNA expression can be induced in MMCs under hypoxia. Interestingly, AS-shRNA-lipid derived fluorescence was observed in normoxic as well as in hypoxic MRMECs, indicating that HIF-1α mRNA can be constitutively expressed in cultured MRMECs and remain unchanged after hypoxia treatment (FIG. 24). These observations are consistent with previously reported results in vascular endothelial cells. Thus, AS-shRNA-lipid can be used for imaging endogenous mRNA in cultured retinal cell and other cell types. Furthermore, a non-sense probe, (NS-shRNA-lipid conjugate) showed minimal fluorescence in hypoxic cells as shown in FIG. 20. The NS-shRNA-lipid probe was designed and synthesized using scrambled sequence of the anti-sense probe with same G-C content as confirmed from a series of MS-data as shown in FIG. 23.

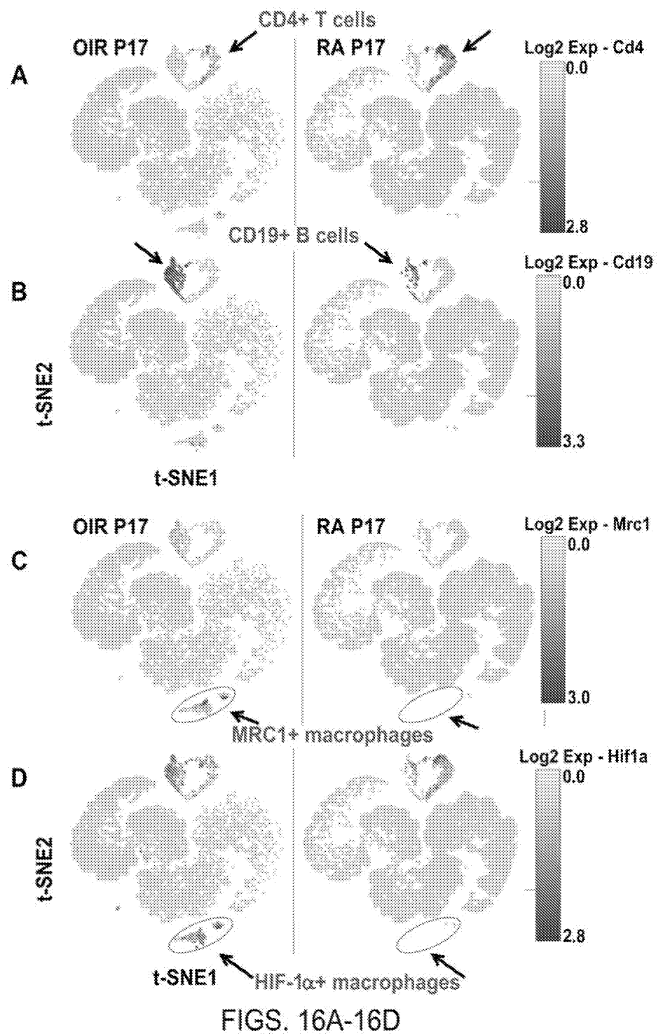

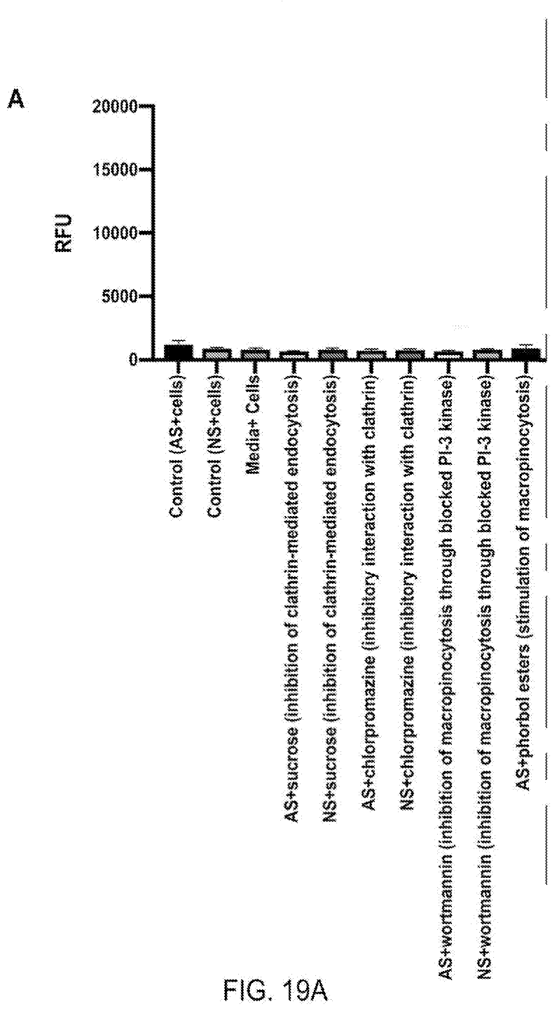

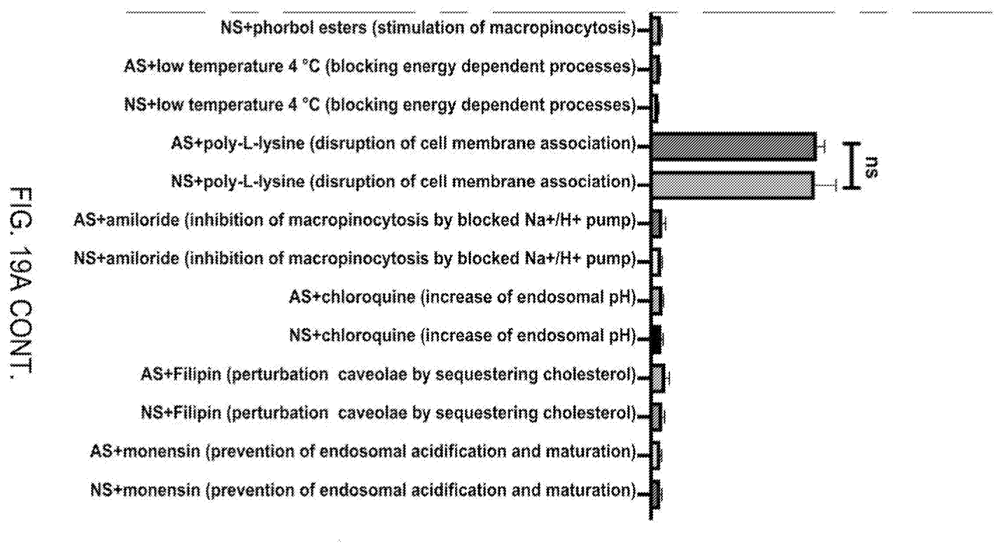

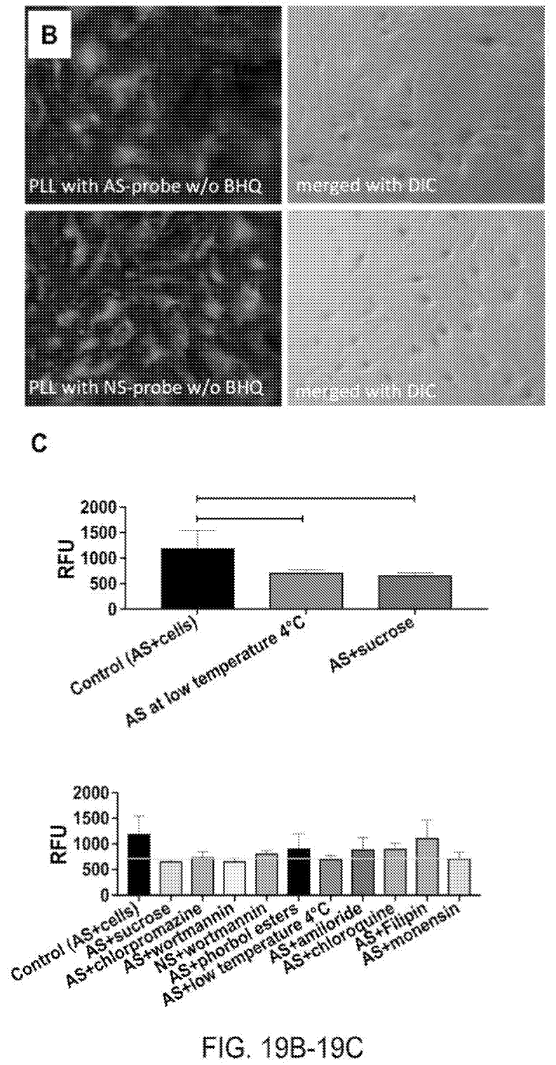

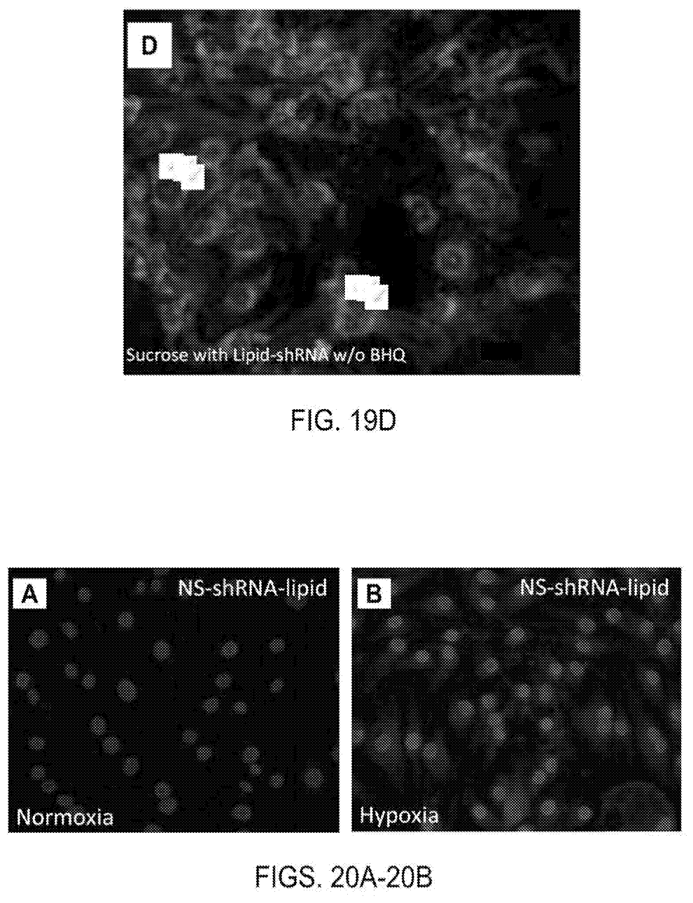

Myeloid-specific HIF-1α expression in mouse oxygen-induced retinopathy (OIR). In an effort to characterize the active cell population and associated HIF-1α expression profiles, single cell RNA sequencing (scRNAseq) data analysis was used to identify a very small number of MRC-1 positive activated macrophages (<1%) in blood samples from oxygen-induced retinopathy (OIR) (FIG. 16C). HIF-1α expression was associated with these MRC-1 positive macrophages in mouse OIR. Also, the MRC-1 positive cells were minimally present in healthy controls. These observations are consistent with previously reported results describing the association of HIF-1α with myeloid-specific progenitor cells. Interestingly, the number of CD4 positive T cells increased in OIR (FIG. 16A). On the other hand, CD19 positive B cells are higher in OIR compared to normal healthy controls (FIG. 16B). These observations are indicative of poor immune system in OIR animals, which can represent poor immune system in human ROP patients due to high oxygen treatment in their early life. Furthermore, most of the CD4 positive T cells and CD19 positive B cells are also positive for HIF-1α in OIR as well as in room air control animals (FIG. 16D). However, AS-shRNA-lipid derived fluorescence were not observed in control retinas as shown in FIG. 19, supporting the possibility of migration of the MRC-1 positive cells into the retina in response to neovascularization.

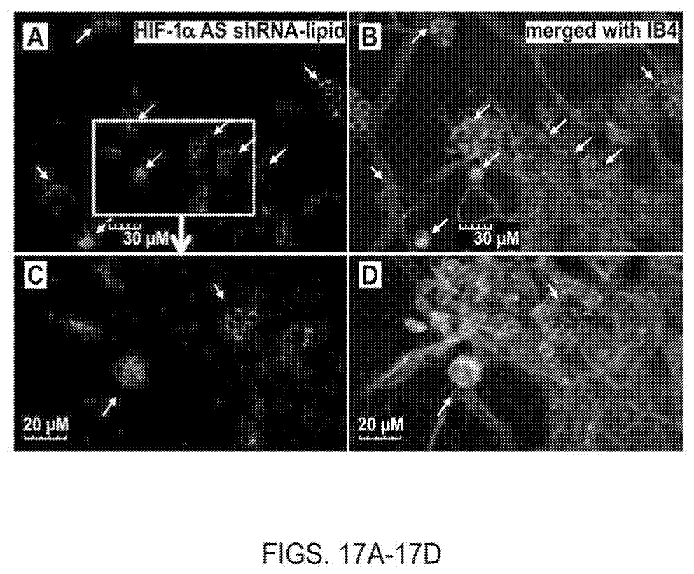

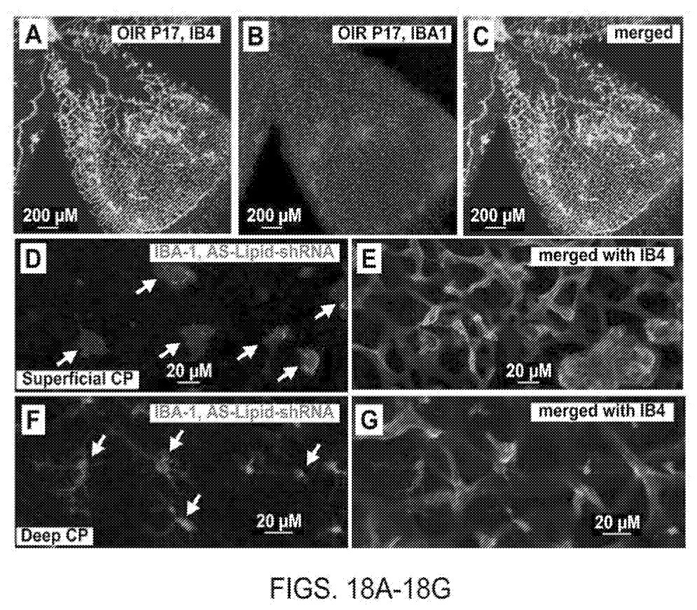

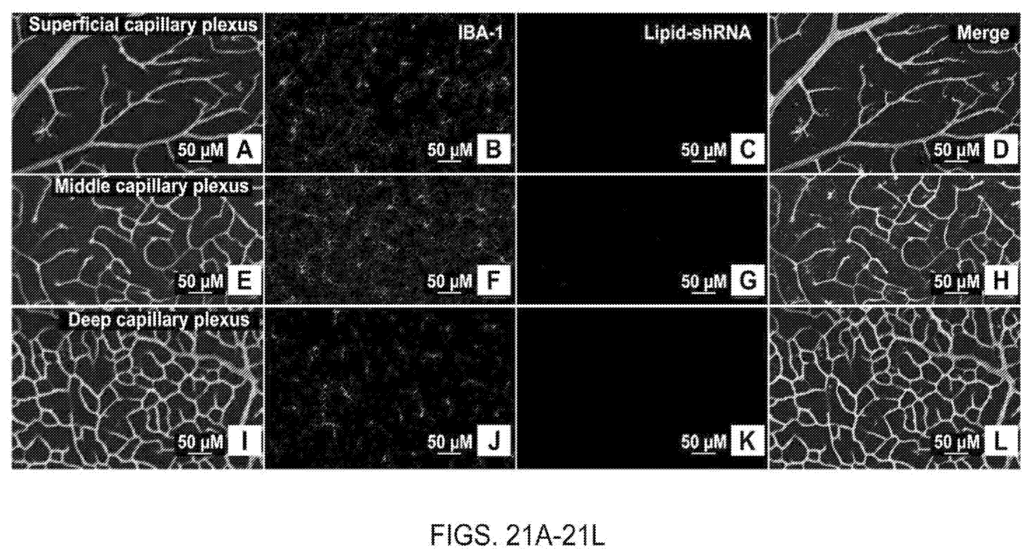

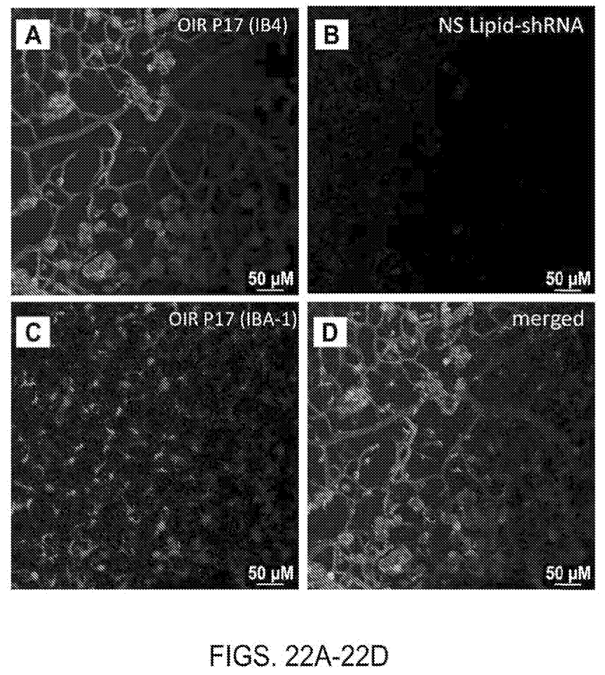

Imaging HIF-1α mRNA using AS-shRNA-lipid in mouse OIR retina. After confirming the association of HIF-1α mRNA with MRC-1 positive myeloid cells in mouse OIR, the newly synthesized AS-shRNA-lipid conjugate were used to track bone marrow derived cells in the OIR retina (FIG. 17). After intraperitoneal injection in OIR animals, AS-shRNA-lipid yielded a strong punctate fluorescence in cells that were associated with neovascularization, due to hybridization with HIF-1α mRNA. Reports have shown that transplanted bone marrow derived myeloid progenitor cells differentiated into microglia in the OIR retina. Based on these observations, the AS-shRNA-lipid associated fluorescence was co-localized with markers of activated cells both in the OIR and room air (RA) control retinas; IBA-1 was used as a marker for retinal microglial cells (FIG. 18 and FIG. 21). Two morphologically distinct microglial populations were observed in the OIR retina; activated microglia reside on the surface of the superficial capillary plexus of the OIR retina, whereas resting microglia resides in the deep capillary plexuses (FIG. 18). Furthermore, AS-shRNA-lipid-derived fluorescence was observed in microglia that resides on superficial capillary plexus (FIGS. 18D-18E). No AS-shRNA-lipid derived fluorescence was observed in ramified IBA-1-positive cells that resided around the deep capillary plexus of the OIR retina as shown in FIGS. 18F-18G. Even though, the expression of HIF-1α in CD4 positive T cells and CD19 positive B cells was observed in room air control animals, no AS-shRNA-lipid derived fluorescence was observed in these control retinas as shown in FIG. 21. Furthermore, no AS-shRNA-lipid-derived fluorescence was observed in the retinal microvascular endothelial cells or in other cell-types that might also be positive for HIF-1α mRNA indicating the possibility of probe-internalization outside of the retina and transported to the retina in response to neovascularization in OIR animals Phagocytic behavior of the activated cells is important for probe internalization. Thus, after intraperitoneal injection, shRNA-lipid conjugates can be internalized into the IBA-1-positive activated cells in OIR animals and then migrated into the retina. This migration can be a response to neovascularization and can be used as measure for severity of retinopathy and disease progression as well as treatment response. Furthermore, a non-sense probe, (NS-shRNA-lipid conjugate) showed minimal fluorescence in the OIR retina as shown in FIG. 22. The NS-shRNA-lipid probe was designed and synthesized from scrambled sequence of the anti-sense probe and thus has a similar molecular mass as shown in FIG. 23.

To characterize pathway mechanism for delivery of AS(or NS)-shRNA-lipid conjugates into primary retinal cells, inhibitors or stimulators of endocytosis or macropinocytosis were examined as shown in FIG. 19. For this assay, both AS- and NS-shRNA-lipid conjugates were synthesized using only the fluorescence dye (Cy5) at the 5′ end, without the black hole quencher (BHQ). Cells were exposed to different inhibitors and stimulators at different concentrations as shown in Table 1: sucrose (inhibition of clathrin-mediated endocytosis), chlorpromazine (inhibitory interaction with clathrin), filipin (perturbation caveolae by sequestering cholesterol), wortmannin (inhibition of macropinocytosis through blocked PI-3 kinase), amiloride (inhibition of macropinocytosis by blocked Na+/H+ pump), phorbol esters (stimulation of macropinocytosis), monensin (prevention of endosomal acidification and maturation) and chloroquine (increase of endosomal pH). In addition, two non-specific inhibitors were also utilized: poly-L-lysine (disruption of cell membrane association), and low temperature (blocking energy dependent processes). Cells were exposed to these treatments for 30 min prior to addition of the shRNA-lipid conjugates. Fluorescence measurements were performed two hours post-incubation with shRNA-lipid. It was observed that poly-L-lysine (PLL) induces shRNA-lipid internalization and the internalization is independent of the sequence present in the shRNA-lipids (FIG. 19B). In addition, also it was also observed that shRNA-lipid internalization is independent of the macropinocytosis pathways as shown in FIG. 19C. Endocytosis was inhibited in presence of sucrose suggesting that the internalization can be clathrin-mediated pathway and are independent caveolae pathways in vitro. However, internalization of shRNA-lipid into the macrophages can be influence by phagocytic behavior of these cells in vivo.

### DISCUSSION

Macrophages were observed in the retina during hyaloid degeneration and in response to neovascularization such as that occurring in proliferative diabetic retinopathy. Regulated expression of HIF-1α by macrophages was demonstrated more than a decade ago. In addition, HIF-1α can be induced in monocytic-cells differentiated into macrophages, indicating that inflammation initiates phenotypic differentiation of monocytes. However, the exact role of macrophages in neovascularization is largely unknown. It is known that tissue macrophages play a key role to promote vasculogenesis as well as angiogenesis. These observations indicate that molecular imaging of specific mRNA biomarkers in activated microglia and macrophages can uncover the role of these cells in the pathogenesis of proliferative retinopathy.

Small interfering RNA (siRNA), antisense-DNA and micro RNA technologies are attractive for mRNA interference. However, these agents have short half-lives and often require toxic transfection reagents. Therefore, these methods are not suitable for real-time in vivo imaging of mRNA. The current study has used AS-shRNA-lipid for molecular imaging of mRNA in mouse OIR without using any added transfection reagents. For diagnostic purposes, topical or systemic delivery of shRNA is vital for clinical applications. Part of the strategy for the application of shRNA-lipid conjugates stems from the albumin binding capacity conferred by the lipid moiety. Albumin is the most abundant serum protein (>40 mg/mL) and has a circulation half-life of about 20 days, making it a natural chaperone for systemic delivery of shRNA conjugates. The data indicate that this chaperone activity greatly facilitates delivery of shRNA-lipid conjugates to target tissues.

In summary, a novel method has been developed to track specific cell populations in ocular tissues using antisense short-hairpin RNA conjugated to diacyl-lipids (AS-shRNA-lipid). Molecular imaging of inflammatory cytokines and tracking of specific cell populations allows physicians to predict neovascularization, a common complication observed in proliferative retinopathies.

### Methods

All chemicals were purchased form Sigma-Aldrich (St. Louis, Mo.) and used as received unless otherwise noted. The mouse primary retinal microvascular endothelial cells (MRMEC) were obtained from Cell Biologics Inc (IL, USA). Primary mouse Müller cells (MMC) were isolated from adult C57BL/6 mice following a previously described method. Custom designed 2′-O-methyl-protected short hairpin RNA (shRNA) and custom oligonucleotides were purchased from Integrated DNA Technologies Inc. (IA, USA).

Design and synthesis of AS-HIF-1α-(or NS)-shRNA-lipid conjugates: The shRNA was computationally designed via energy minimization to achieve the formation of the hairpin structure. Each of the shRNA-oligonucleotides was coupled to a Cy3 dye (fluorophore) and C6 amino group to facilitate conjugation to the diacyl-lipid. The 3′ end was coupled to a BHQ2. The 2′-O-methyl-protected short hairpin RNA (shRNA) that incorporate anti-sense sequence complementary to mouse HIF-1α mRNA, position 2327 to 2349 (NM_010431.2) was synthesized and purified using HPLC system. A scrambled sequence the anti-sense sequence (NS-shRNA) was also synthesized and characterized using MS-analysis. The anti-sense sequences were extensively BLAST searched to determine no significant overlap with any other mouse mRNA sequence. The same was performed on the non-sense sequence to confirm non-specific binding. The anti-sense and the non-sense sequences are located within the loop of the hairpin structure as shown in FIG. 14. A self-complementary sequence was incorporated to form the stem of the shRNA hairpin. Finally, the shRNA was conjugated to the diacyl-lipid according to a previously described method. Excess reagents were removed by centrifugal filtration using a filter with a 10K molecular weight cut-off (Amicon Ultracel 10K from Millipore, Billerica, Mass.). The freshly conjugated lipid-oligonucleotides were washed three times with PBS (Life Technologies Corporation; Grand Island, N.Y.) and stored at 4° C. until used. The shRNA-lipid conjugates can spontaneously form spherical nanoparticles.

Dynamic light scattering (DLS): DLS measurements were performed on a Malvern Zetasizer Nano ZS (Malvern Instruments, Inc.; Westborough, Mass.). Particle measurements were performed at a concentration of 10 μM shRNA-lipid in PBS (Life Technologies Corp.; Carlsbad, Calif.). These measurements were performed in triplicate.

Transmission electron microscopy (TEM): shRNA-lipid probes were mounted on 300-mesh copper grids and stained with 2% uranyl acetate. Samples were subsequently imaged on the Philips/FBI Tecnai T12 electron microscope (Hillsboro, Oreg.) at various magnifications.

Animals: Multi-timed pregnant C57BL/6J female mice were purchased from Charles River Laboratories. All animal procedures used in this study were approved by the Vanderbilt University Institutional Animal Care and Use Committee and were performed in accordance with the Association for Research in Vision and Ophthalmology Statement for the Use of Animals in Ophthalmic and Vision Research.

Primary cell culture: MRMECs were cultured in T-75 cell culture flasks (Thermo Fisher Scientific; Wilmington, Mass.) coated with attachment factor (Cell Systems; Danvers, Mass.) and in growth medium consisting of endothelial basal medium (EBM; Lonza; Walkersville, Md.) supplemented with 2% FBS (Lonza) and endothelial cell growth supplements (EGM SingleQuots; Lonza). Primary MMCs were cultured in T-75 cell culture flasks (Thermo Fisher Scientific; Wilmington, Mass.) coated with attachment factor (Cell Systems; Danvers, Mass.) and in low glucose DMEM growth medium consisting of 10% FBS (Lonza), 1× GlutaMAX and 1× penicillin-streptomycin. All cultures were incubated at 37° C., 5% CO2 and 95% relative humidity (20.9% oxygen). The cells were cultured in 95-well plates and treated with shRNA-lipid in complete growth medium. Hypoxia was induced following the previously described method. Briefly, cells were treated with shRNA-lipid diluted in complete media and the assay plates were placed into a humidified hypoxic chamber. Ambient air was displaced with a mixture of 5% CO₂ and 95% N2 at a flow rate of 20 L/min for 5 min according to manufacturer instructions and published methods. The chamber was clamped and placed at 37° C. for the appropriate treatment time. For probe internalization assays, cells were treated with the inhibitors or stimulators for 30 minutes with doses as shown in Table 1. After the treatment, cells were treated with shRNA-lipid in complete cell culture medium, for two hours and then a plate-based fluorescence assay was performed to analyze intracellular delivery.

Ex vivo imaging of mRNA in mouse OIR: To generate the OIR mouse model, dams with their pups were treated with 75% oxygen for 5 days from postnatal day 7 (P7) to P12. On P12, pups were removed from the hyperoxic chamber and stayed with the nursing mother in normal air condition for additional 5 days. At P17 AS-(or NS)-shRNA-lipid conjugates in sterile saline were injected intraperitoneally at a dose of 0.5 mg/kg. After 18 hours, AS-(or NS)-shRNA-lipid dependent fluorescence imaging was performed ex vivo. Briefly, animals were sacrificed, enucleated and the globes were fixed in 10% neutral buffered formalin (NBF). Retinas were dissected and blocked/permeabilized in 10% donkey serum with 1% Triton X-100 and 0.05% Tween 20 in TBS for 2 hours and were then counter-stained for IB4 and IBA-1 conjugated to Alexafluor-dyes (Life Technologies; Grand Island, N.Y.). The tissues were then mounted with Prolong Gold mounting medium with DAPI (Life Technologies; Grand Island, N.Y.). Images were taken using an epifluorescence Nikon Eclipse Ti-E inverted microscope (Melville, N.Y.).

Isolation and single cell RNAseq analysis of bone marrow-derive cells from mouse OIR and RA pups: Peripheral blood samples were collected from deeply anesthetized OIR and age-matched RA control pups. Bone marrow-derive mononuclear cells were isolated from fresh blood within 2 hours of collection, using Ficoll-Paque density gradient centrifugation as described. Each sample (targeting 5,000 cells/sample) was processed for single cell 5′ RNA sequencing utilizing the 10× Chromium system. Libraries were prepared using P/N 1000006, 1000080, and 1000020 following the manufacturer's protocol. The libraries were sequenced using the NovaSeq 6000 with 150 bp paired end reads. RTA (version 2.4.11; Illumina) was used for base calling and analysis was completed using 10× Genomics Cell Ranger software v2.1.1. or Loupe Cell Browser.

Statistics. Data were expressed as mean±% SDM and statistical differences among groups were determined by one-way analysis of variance (ANOVA) using Prism 6 (Graph-Pad, San Diego, Calif.) followed by Bonferroni post hoc test to determine significant differences between specific groups. A ‘p’ value <0.05 was considered statistically significant.

TABLE 1
Influence of Inhibitor/activator on intracellular
delivery of shRNA-lipid.

| Treatment | Concentration* | | Effect |
|---|---|---|---|
| Chlorpromazine | 10 | μM | inhibitory interaction with clathrin |
| Hydrochloride | | | |
| Filipin | 5 | μg/mL | perturbation caveolae by |
| | | | sequestering cholesterol |
| Sucrose | 100 | mM | inhibition of clathrin-mediated |
| | | | endocytosis |
| Wortmannin | 500 | nM | inhibition of macropinocytosis |
| | | | through blocked PI-3 kinase |
| Amiloride | 10 | μM | inhibition of macropinocytosis by |
| Hydrochloride | | | blocked Na+/H+ pump |
| Phorbol ester | 10 | nM | stimulation of macropinocytosis |
| Monensin | 5 | μM | prevention of endosomal |
| | | | acidification and maturation |
| Chloroquine | 100 | μM | increase of endosomal pH |
| poly-L-lysine | 10 | nM | disruption of cell membrane |
| | | | association |

| | | |
|---|---|---|
| Low temperature | — | blocking energy dependent processes |
| (4° C.) | | |

*Concentrations used in this study.

SEQUENCES
In the following sequences: mG means 2′-MeO
protected G, mC means 2′-MeO protected C,
mA means 2′-MeO protected A, mT means 2′-MeO
protected T, mU means 2′-MeO protected
U; 2′-MeO means 2′-O-methyl.
(MI-9-2018-mENG seq-2 Cy3)
SEQ ID NO: 1
mGmCmAmGmCmAmCmUmGmUmGmAmUmGmUmUmGmAmCmUmCmUmUmGmG
mCmGmCmUmGmC
(MI-2017-mHIF-1a Cy3)
SEQ ID NO: 2
mCmCmGmGmUmAmUmUmGmUmCmCmUmUmCmGmUmCmUmCmUmGmUmUmU
mUmUmGmAmCmCmGmG
SEQ ID NO: 3
GCAGCTCTGTCTTTCTTTGGTCTGCGCTGC
SEQ ID NO: 4
TTGCAGACCAAAGAAAGACAGATT
(MI-9-2018-mENG seq-1 Cy3)
SEQ ID NO: 5
mGmCmAmGmCmUmGmCmAmAmCmUmCmAmGmUmUmCmCmAmUmCmAmUmU
mAmCmGmGmGmCmUmGmC
(MI-9-2018-mENG seq-3 Cy3)
SEQ ID NO: 6
mGmCmUmCmGmUmUmUmGmAmCmCmUmUmGmCmUmUmCmCmUmGmGmAmA
mAmGmAmUmCmGmAmGmC
SEQ ID NO: 7
GCCAAGAGTCAACATCACAGTGCT
SEQ ID NO: 8
CCGTAATGATGGAACTGAGTTGCA
SEQ ID NO: 9
ATCTTTCCAGGAAGCAAGGTCAAA
SEQ ID NO: 10
(MI-9-2018-NSense Cy3)
mCmCmGmGmUmUmUmAmGmUmUmCmCmUmGmUmUmCmUmGmUmUmGmUmC
mUmUmCmAmCmCmGmG
SEQ ID NO: 11
mGmCmAmGmCmUmCmUmGmUmCmUmUmUmCmUmUmUmGmGmUmCmUmGmC
mGmCmUmGmC
SEQ ID NO: 12
TTGCCAAGAGTCAACATCACAGTGCTT
SEQ ID NO: 13
TTG CCA AGA GTC AAC ATC ACA GTG CTT
SEQ ID NO: 14
TTATCTTTCCAGGAAGCAAGGTCAAATT
SEQ ID NO: 15
TTGAAGACAACAGAACAGGAACTAATT
SEQ ID NO: 16
TTGAAGACAACAGAAGAGGAACTAATT
SEQ ID NO: 17
mGmCmAmGmCmUmGmCmAmAmCmUmCmAmGmUmUmCmCmAmUmCmAmUmU
mAmCmGmGmGmCmUmGmC
SEQ ID NO: 18
CAAAAACAGAGACGAAGGACAAT
SEQ ID NO: 19
GCAGACCAAAGAAAGACAGA
SEQ ID NO: 20
GCAGACCGAAGAAAGACAGA

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

1. A probe for the detection of an RNA, comprising:
a short hairpin RNA sequence (shRNA), wherein the shRNA sequence comprises an anti-sense sequence complementary to a target sequence of the RNA;
a lipid moiety conjugated to the shRNA;
a quencher conjugated to the shRNA; and
a fluorescent dye conjugated to the shRNA.

2. The probe of claim 1, where the RNA comprises an endoglin mRNA.

3. The probe of claim 1, where the shRNA sequence comprises SEQ ID NO:1.

4. The probe of claim 1, where the RNA comprises a HIF-1α mRNA.

5. The probe of claim 1, where the shRNA sequence comprises SEQ ID NO:2.

6. The probe of claim 1, where the shRNA sequence comprises about 15-45 nucleotides.

7. The probe of claim 1, wherein the target sequence of the RNA is about 21 nucleotides.

8. The probe of claim 1, where the shRNA comprises at least one chemically modified nucleotide.

9. The probe of claim 8, where the at least one chemically modified nucleotide comprises 2′-O-methyl (2′MeO).

10. The probe of claim 1, where the lipid moiety is a diacyl lipid moiety.

11. The probe of claim 1, where the lipid moiety is conjugated to the shRNA by a linker.

12. The probe of claim 1, where the lipid moiety is conjugated to the shRNA by a polyethylene glycol (PEG) linker.

13. The probe of claim 1, where the quencher is BHQ-2.

14. The probe of claim 1, where the fluorescent dye is cyanine-3 (Cy3).

15. A method for detecting an RNA, comprising:
introducing the probe of claim 1 into a cell or a tissue;
allowing the probe to bind the target sequence; and
detecting the fluorescent dye after the probe binds to the target sequence.

16. The method of claim 15, wherein the cell or tissue is an ocular cell or tissue.

17. A method for detecting neovascularization, comprising:
introducing the probe of claim 1 into a cell or a tissue;
allowing the probe to bind the target sequence; and
detecting the fluorescent dye after the probe binds to the target sequence.

18. The method of claim 17, wherein the cell or tissue is an ocular cell or tissue.

19. A method for treating neovascularization, comprising:
detecting neovascularization in a subject, comprising:
introducing the probe of claim 1 into a cell or a tissue;
allowing the probe to bind the target sequence; and
detecting the fluorescent dye after the probe binds to the target sequence; and
administering to the subject an effective amount of an inhibitor of neovascularization, if neovascularization is detected in the subject.

20. The method of claim 19, wherein the inhibitor of neovascularization is selected from bevacizumab or ranibizumab.
